(19) United States
(12) Patent Application Publication
(10) Pub. No.: US 2022/0380822 A1
(43) Pub. Date: Dec. 1, 2022

(54) APPLICATION OF BRANCHED-CHAIN A-KETOACID DEHYDROGENASE COMPLEX IN PREPARATION OF MALONYL COENZYME A

(71) Applicant: INSTITUTE OF MICROBIOLOGY CHINESE ACADEMY OF SCIENCES, Beijing (CN)

(72) Inventors: Weifeng LIU, Beijing (CN); Bo LIU, Beijing (CN); Qianqian CUI, Beijing (CN); Yong TAO, Beijing (CN)

(21) Appl. No.: 17/635,178

(22) PCT Filed: Aug. 20, 2020
(86) PCT No.: PCT/CN2020/110232

§ 371 Date: Jul. 22, 2022

(30) Foreign Application Priority Data
Aug. 23, 2019 (CN) 201910782183.X

(51) Int. Cl. C12P 19/32; C12N 15/70; C12N 1/20; C12N 9/10; C12P 7/22

(52) U.S. Cl. CPC C12P 19/32; C12N 15/70; C12N 1/205; C12N 9/1029; C12P 7/22; C12Y 102/01075

(57) ABSTRACT

An application of a branched-chain α-ketoacid dehydrogenase complex in preparation of malonyl coenzyme A. A method for preparing malonyl-CoA using a branched-chain α-ketoacid dehydrogenase complex, the method comprising introducing a gene encoding a branched-chain α-ketoacid dehydrogenase complex into a biological cell strain to obtain a recombinant cell strain capable of expressing the gene encoding the branched-chain α-ketoacid dehydrogenase complex; culturing the recombinant cell strain to prepare malonyl-CoA; the branched-chain α-ketoacid dehydrogenase complex is the following M1) or M2): M1) a set of proteins consisting of a bkdF protein, a bkdG protein, a bkdH protein and a lpdA1 protein; M2) a set of proteins consisting of a bkdA protein, a bkdB protein, a bkdC protein and the lpdA1 protein. Experimental results show that by using the branched-chain α-ketoacid dehydrogenase complex, not only malonyl-CoA can be prepared, but also a target product using malonyl-CoA as an intermediate product can further be prepared.

### TECHNICAL FIELD

The present invention relates to application of branched-chain α-ketoacid dehydrogenase complex in preparation of malonyl coenzyme A in the field of biotechnology.

### BACKGROUND OF THE INVENTION

Flavonoids are widely distributed in natural plants and are a class of compounds containing the structure of 2-phenylchromone, which belong to the secondary metabolites of plants. Flavonoids have anti-free radical and anti-oxidation effects, and at the same time, these compounds also have antibacterial, anti-tumor and immune-enhancement activities. Polyketides are also a class of important secondary metabolites, which are formed by bacteria, fungi, actinomycetes or plants through continuous decarboxylation and condensation reactions of lower carboxylic acids such as acetic acid, malonic acid, butyric acid, etc. The synthetic pathway of polyketides is similar to long-chain fatty acids. Polyketides are widely used as antibiotics in clinical practice because of their important biological activities, such as erythromycin, the anticancer drug doxorubicin, the antifungal agent amphotericin, the antiparasitic agent avermectin, the insecticide spinosad and the immunosuppressant rapamycin.

In industry, polyketides are mainly produced by their natural producing bacterium Streptomyces, but the production of polyketides using Streptomyces faces the problems of complex regulation of production strains and difficulty in increasing yield. Attempting to synthesize polyketides using Escherichia coli with a clear genetic background as a chassis cell is not only conducive to the realization of high-level synthesis of target compounds, but also helps to clarify the synthesis and regulation mechanisms of polyketides. At present, polyketides such as erythromycin have been successfully synthesized in Escherichia coli, but their yields are still low. The main reason is that the synthesis of polyketides is limited by the content of intracellular malonyl-CoA. Malonyl-CoA is also an important precursor for the synthesis of flavonoids, and the biosynthesis of flavonoids is also limited by the content of malonyl-CoA.

Increasing the intracellular level of malonyl-CoA is a major work to improve the production of polyketides, flavonoids, and other malonyl-CoA-derived chemicals in the biotechnological fields. Acetyl-CoA is believed the only precursor of malonyl-CoA. In the central metabolic pathway, acetyl-CoA is mainly provided from pyruvic acid, which can be supplied from glucose through glycolysis pathway. Most of acetyl-CoA enters the tricarboxylic acid cycle, and a small amount of acetyl-CoA is involved in the synthesis of fatty acids. Malonyl-CoA is the direct precursor of fatty acid synthesis and is obtained by acetyl-CoA carboxylase catalyzing acetyl-CoA. This step is an energy-consuming step. And the reaction catalyzed by acetyl-CoA carboxylase involves fixation of CO₂, making it difficult to optimize by metabolic engineering strategies. At the same time, in Escherichia coli, in order to balance different pathways, including the synthesis of phospholipids, fatty acids, and cell growth, the intracellular concentration of malonyl-CoA is usually controlled at a very low level. In order to improve the malonyl-CoA levels, many current researches focus on the field of metabolic flux regulation, and the production of malonyl-CoA in cells is increased through the metabolic engineering technology of multi-target genetic manipulation, such as the strategies of increasing the expression level of key enzyme acetyl-CoA carboxylase, knocking out the competitive branches of acetyl-CoA and malonyl-CoA, etc.

### SUMMARY OF THE INVENTION

The purpose of the present invention is to provide a new function of the branched-chain α-ketoacid dehydrogenase complex. The branched-chain α-ketoacid dehydrogenase complex can catalyze the formation of malonyl-CoA from the substrate oxaloacetate.

The present invention first provides a method for preparing malonyl-CoA, the method comprising the following steps 11) and 12):

11) introducing a gene encoding a branched-chain α-ketoacid dehydrogenase complex into a biological cell strain to obtain a recombinant cell strain capable of expressing the gene encoding the branched-chain α-ketoacid dehydrogenase complex, which was named recombinant cell strain A;

12) culturing the recombinant cell strain A to prepare malonyl-CoA.

In the above method, the branched-chain α-ketoacid dehydrogenase complex can be the following M1) or M2):

M1) a set of proteins consisting of a bkdF protein (branched-chain α-ketoacid dehydrogenase E1α subunit), a bkdG protein (branched-chain β-ketoacid dehydrogenase E1β subunit), a bkdH protein (branched-chain α-ketoacid dehydrogenase E2 subunit) and a lpdA1 protein (branched-chain α-ketoacid dehydrogenase E3 subunit);

M2) a set of proteins consisting of a bkdA protein (branched-chain α-ketoacid dehydrogenase E1α subunit), a bkdB protein (branched-chain β-ketoacid dehydrogenase E1β subunit), a bkdC protein (branched-chain α-ketoacid dehydrogenase E2 subunit) and the lpdA1 protein;

the gene encoding the branched-chain α-ketoacid dehydrogenase complex can be the following L1) or L2):

L1) a set of genes consisting of a gene encoding the bkdF protein, a gene encoding the bkdG protein, a gene encoding the bkdH protein and a gene encoding the lpdA1 protein;

L2) a set of genes consisting of a gene encoding the bkdA protein, a gene encoding the bkdB protein, a gene encoding the bkdC protein and a gene encoding the lpdA1 protein.

In the above method, the bkdF protein, the bkdG protein, the bkdH protein, the lpdA1 protein, the bkdA protein, the bkdB protein and the bkdC protein and their encoding genes can be derived from Streptomyces avermitilis.

In the above methods, the bkdF protein can be the following a1) or a2) protein:

a1) a protein having the amino acid sequence shown in SEQ ID NO: 10 in the sequence listing;

a2) a protein having 75% or more identity with and the same function as the amino acid sequence shown in SEQ ID NO: 10, which is obtained by performing substitution and/or deletion and/or addition of one or more amino acid residues on the amino acid sequence shown in SEQ ID NO: 10 in the sequence listing.

The bkdG protein can be the following a3) or a4) protein:

a3) a protein having the amino acid sequence shown in SEQ ID NO: 1 in the sequence listing;

a4) a protein having 75% or more identity with and the same function as the amino acid sequence shown in SEQ ID NO: 11, which is obtained by performing substitution and/or deletion and/or addition of one or more amino acid residues on the amino acid sequence shown in SEQ ID NO: 11 in the sequence listing.

The bkdH protein can be the following a5) or a6) protein:

a5) a protein having the amino acid sequence shown in SEQ ID NO: 12 in the sequence listing;

a6) a protein having 75% or more identity with and the same function as the amino acid sequence shown in SEQ ID NO: 12, which is obtained by performing substitution and/or deletion and/or addition of one or more amino acid residues on the amino acid sequence shown in SEQ ID NO: 12 in the sequence listing.

The lpdA1 protein can be the following a7) or a8) protein:

a7) a protein having the amino acid sequence shown in SEQ ID NO: 13 in the sequence listing;

a8) a protein having 75% or more identity with and the same function as the amino acid sequence shown in SEQ ID NO: 13, which is obtained by performing substitution and/or deletion and/or addition of one or more amino acid residues on the amino acid sequence shown in SEQ ID NO: 13 in the sequence listing.

The bkdA protein can be the following a9) or a10) protein:

a9) a protein having the amino acid sequence shown in SEQ ID NO: 7 in the sequence listing;

a10) a protein having 75% or more identity with and the same function as the amino acid sequence shown in SEQ ID NO: 7, which is obtained by performing substitution and/or deletion and/or addition of one or more amino acid residues on the amino acid sequence shown in SEQ ID NO: 7 in the sequence listing.

The bkdB protein can be the following a11) or a12) protein:

a11) a protein having the amino acid sequence shown in SEQ ID NO: 8 in the sequence listing;

a12) a protein having 75% or more identity with and the same function as the amino acid sequence shown in SEQ ID NO: 8, which is obtained by performing substitution and/or deletion and/or addition of one or more amino acid residues on the amino acid sequence shown in SEQ ID NO: 8 in the sequence listing.

The bkdC protein can be the following a13) or a14) protein:

a13) a protein having the amino acid sequence shown in SEQ ID NO: 9 in the sequence listing;

a14) a protein having 75% or more identity with and the same function as the amino acid sequence shown in SEQ ID NO: 9, which is obtained by performing substitution and/or deletion and/or addition of one or more amino acid residues on the amino acid sequence shown in SEQ ID NO: 9 in the sequence listing.

In the above methods, the gene encoding the bkdF protein can be the following b1) or b2):

b1) a DNA molecule having the nucleotide sequence shown in positions 1-1221 of SEQ ID NO: 2 in the sequence listing;

b2) a DNA molecule having 75% or more identity with and the same function as the nucleotide sequence defined in b1).

The gene encoding the bkdG protein can be the following b3) or b4):

b3) a DNA molecule having the nucleotide sequence shown in positions 1223-2200 of SEQ ID NO: 2 in the sequence listing;

b4) a DNA molecule having 75% or more identity with and the same function as the nucleotide sequence defined in b3).

The gene encoding the bkdH protein can be the following b5) or b6) or b7):

b5) a DNA molecule having the nucleotide sequence shown in SEQ ID NO: 3 in the sequence listing;

b6) a DNA molecule having the nucleotide sequence shown in positions 2220-3608 of SEQ ID NO: 2 in the sequence listing;

b7) a DNA molecule having 75% or more identity with and the same function as the nucleotide sequence defined in b5) or b6).

The gene encoding the lpdA1 protein can be the following b8) or b9) or b10):

b8) a DNA molecule having the nucleotide sequence shown in SEQ ID NO: 5 in the sequence listing;

b9) a DNA molecule having the nucleotide sequence shown in SEQ ID NO: 4 in the sequence listing;

b10) a DNA molecule having 75% or more identity with and the same function as the nucleotide sequence defined in b8) or b9).

The gene encoding the bkdA protein can be the following b11) or b12):

b11) a DNA molecule having the nucleotide sequence shown in positions 1-1146 of SEQ ID NO: 1 in the sequence listing;

b12) a DNA molecule having 75% or more identity with and the same function as the nucleotide sequence defined in b11).

The gene encoding the bkdB protein can be the following b13) or b14):

b13) a DNA molecule having the nucleotide sequence shown in positions 1220-2224 of SEQ ID NO: 1 in the sequence listing;

b14) a DNA molecule having 75% or more identity with and the same function as the nucleotide sequence defined in b13).

The gene encoding the bkdC protein can be the following b15) or b16):

b15) a DNA molecule having the nucleotide sequence shown in positions 2224-3591 of SEQ ID NO: 1 in the sequence listing;

b16) a DNA molecule having 75% or more identity with and the same function as the nucleotide sequence defined in b15).

In the above methods, the step of “introducing a gene encoding a branched-chain α-ketoacid dehydrogenase complex Into a biological cell strain” can specifically be “introducing an expression vector containing the gene encoding the branched-chain α-ketoacid dehydrogenase complex into the biological cell strain”.

The expression vector can be a plasmid, a cosmid, a phage or virus vector. The plasmid can specifically be pYB1k or pLB1a, the sequence of the plasmid pYB1k is shown in SEQ ID NO: 6 in the sequence listing, and the sequence of the plasmid pLB1a is shown in SEQ ID NO: 24 in the sequence listing.

The four independent genes (the above L1) or L2)) of the gene encoding the branched-chain α-ketoacid dehydrogenase complex can be introduced into the biological cell strain through a co-expression vector containing these genes. The co-expression vector can be pYB1k-bkdABC-lpdA1, pYB1k-bkdFGH-lpdA1 or pYB1k-bkdFG-opbkdH-oplpdA1; the vector pYB1k-bkdABC-lpdA1 is a recombinant vector obtained by inserting the gene encoding the bkdA protein, the gene encoding the bkdB protein, the gene encoding the bkdC protein and the gene encoding the lpdA1 protein into the vector pYB1k and can express the bkdA protein, the bkdB protein, the bkdC protein and the lpdA1 protein: both the vector pYB1k-bkdFGH-lpdA1 and the vector pYB1k-bkdFG-opbkdH-oplpdA1 are recombinant vectors obtained by inserting the gene encoding the bkdF protein, the gene encoding the bkdG protein, the gene encoding the bkdH protein and the gene encoding the lpdA1 protein into the vector pYB1k and can express the bkdF protein, the bkdG protein, the bkdH protein and the lpdA1 protein.

In the above methods, the biological cell strain contains a branched-chain α-ketoacid synthesis pathway, and step 11) can further comprise the step of inhibiting the synthesis of branched-chain α-ketoacids in the biological cell strain.

The obtained recombinant cell strain A contains the gene encoding the branched-chain α-ketoacid dehydrogenase complex, and the synthesis of branched-chain α-ketoacids is inhibited therein.

In the above method, the step of “Inhibiting the synthesis of branched-chain α-ketoacids” can be performed by “knocking out at least one gene in the branched-chain α-ketoacid synthesis pathway in the biological cell strain, or reducing the content or the activity of at least one protein encoded by the genes in the branched-chain α-ketoacid synthesis pathway”.

In the above methods, the step of “inhibiting the synthesis of branched-chain α-ketoacids” can be performed by “knocking out the ilvA gene (threonine deaminase gene) or/and the ilvE gene (branched-chain amino acid transaminase gene) in the biological cell strain, or reducing the content or the activity of the proteins encoded by the ilvA gene or/and the ilvE gene in the biological cell strain”.

The biological cell strain contains the ilvA gene or/and the ilvE gene.

In the above method, the ilvA gene can encode the following a15) or a16) protein:

a15) a protein having the amino acid sequence shown in SEQ ID NO: 15 in the sequence listing;

a16) a protein having 75% or more identity with and the same function as the amino acid sequence shown in SEQ ID NO: 15, which is obtained by performing substitution and/or deletion and/or addition of one or more amino acid residues on the amino acid sequence shown in SEQ ID NO: 15 in the sequence listing.

The ilvE gene can encode the following a17) or a18) protein:

a17) a protein having the amino acid sequence shown in SEQ ID NO: 17 in the sequence listing;

a18) a protein having 75% or more identity with and the same function as the amino acid sequence shown in SEQ ID NO: 17, which is obtained by performing substitution and/or deletion and/or addition of one or more amino acid residues on the amino acid sequence shown in SEQ ID NO: 17 in the sequence listing.

Further,

the ilvA gene can be the following b17) or b18):

b17) a DNA molecule having the nucleotide sequence shown in SEQ ID NO: 14 in the sequence listing;

b18) a DNA molecule having 75% or more identity with and the same function as the nucleotide sequence defined in b17).

The ilvE gene can be the following b19) or b20):

b19) a DNA molecule having the nucleotide sequence shown in SEQ ID NO: 16 in the sequence listing;

b20) a DNA molecule having 75% or more identity with and the same function as the nucleotide sequence defined in b19).

In the above methods, the step of “knocking out the ilvA gene in the biological cell strain” can be performed by homologous recombination, and specifically can be achieved by using Escherichia coli JW3745 strain with the ilvA gene knockout trait.

In the above methods, the step of “knocking out the ilvE gene in the biological cell strain” can be performed by homologous recombination, and specifically can be achieved by using Escherichia coli JW5606 strain with the ilvE gene knockout trait.

In the above methods, step 11) can further comprise the step of introducing a gene encoding a ppc protein (phosphoenolpyruvate carboxylase) into the biological cell strain and expressing the encoding gene, or increasing the content of the ppc protein or enhancing the activity of the ppc protein in the biological cell strain. The obtained recombinant cell strain A contains the gene encoding the branched-chain α-ketoacid dehydrogenase complex and the gene encoding the ppc protein, and the synthesis of branched-chain α-ketoacids is inhibited therein.

Further, the ppc protein and its encoding gene can be derived from Corynebacterium glutamicum.

Still further, the ppc protein can be the following a19) or a20):

a19) a protein having the amino acid sequence shown in SEQ ID NO: 19 in the sequence listing;

a20) a protein having 75% or more identity with and the same function as the amino acid sequence shown in SEQ ID NO: 19, which is obtained by performing substitution and/or deletion and/or addition of one or more amino acid residues on the amino acid sequence shown in SEQ ID NO: 19 in the sequence listing.

The gene encoding the ppc protein can be the following b21) or b22):

b21) a DNA molecule having the nucleotide sequence shown in SEQ ID No: 18 in the sequence listing;

b22) a DNA molecule having 75% or more identity with and the same function as the nucleotide sequence defined in b21).

In the above methods, the biological cell strain can express outer membrane protease VII, and step 11) can further comprise the step of knocking out the gene encoding the outer membrane protease VII in the biological cell strain or reducing the content or the activity of the outer membrane protease VII in the biological cell strain.

Further, the outer membrane protease VII can be an ompT protein.

Still further, the ompT protein is the following a21) or a22):

a21) a protein having the amino acid sequence shown in SEQ ID NO: 28 in the sequence listing;

a22) a protein having 75% or more identity with and the same function as the amino acid sequence shown in SEQ ID NO: 28, which is obtained by performing substitution and/or deletion and/or addition of one or more amino acid residues on the amino acid sequence shown in SEQ ID NO: 28 in the sequence listing.

The gene encoding the ompT protein is the following b23) or b24):

b23) a DNA molecule having the nucleotide sequence shown in SEQ ID NO: 27 in the sequence listing;

b24) a DNA molecule having 75% or more identity with and the same function as the nucleotide sequence defined in b23).

In the above methods, the step of “introducing a gene encoding a ppc protein into the biological cell strain” can specifically be “introducing an expression vector containing the gene encoding the ppc protein into the biological cell strain”, or can be achieved by “recombining the gene encoding the ppc protein into the genome of the biological cell strain and expressing the gene encoding the ppc protein”.

In the above methods, the biological cell strain can contain oxaloacetate synthesis pathway and can synthesize oxaloacetate.

Further, the biological cell strain can be a microbial cell strain, an animal cell strain or a plant cell strain.

Still further, the microbial cell strain can be N1) or N2) or N3): N1) bacteria or fungi; N2) Escherichia coli; N3) Escherichia coli BW25113.

The present invention also provides a method for preparing malonyl-CoA, the method comprising: using the branched-chain α-ketoacid dehydrogenase complex to carry out a catalytic reaction to obtain malonyl-CoA from the substrate oxaloacetate.

In the above method, the catalytic reaction can be carried out in buffer F; the buffer F is composed of a solvent and solutes, and the solvent is 50 mM Tris-HCl buffer (pH=7.0), and the solutes and their concentrations in the buffer F are as follows: 0.1 mM coenzyme A, 0.2 mM dithiothreitol, 0.2 mM triphenyl phosphate, 1 mM MgSO₄, and 2 mM NAD⁺ (oxidized nicotinamide adenine dinucleotide).

The catalytic reaction can be carried out at 30-37° C. Further, the catalytic reaction can be carried out at 30° C.

The present invention also provides a method for producing a target product with malonyl-CoA as an intermediate product, the method comprising: culturing the recombinant cell strain A to prepare the target product.

In the above method, the target product can be 3-hydroxypropionic acid, and the method comprises the steps of: introducing into the recombinant cell strain A a gene encoding a mcr protein (malonyl-CoA reductase) and expressing the encoding gene or increasing the content of the mcr protein or enhancing the activity of the mcr protein in the recombinant cell strain A, to obtain a recombinant cell strain, which is recorded as recombinant cell strain-mcr; culturing the recombinant cell strain-mcr to prepare the target product.

Further, the mcr protein and its encoding gene can be derived from Chloroflexus aurantiacus.

Still further, the mcr protein can be composed of an mcr N-terminal domain and an mcr C-terminal domain, and the mcr N-terminal domain can be the following a23) or a24):

a23) a protein having the amino acid sequence shown in SEQ ID NO: 22 in the sequence listing;

a24) a protein having 75% or more identity with and the same function as the amino acid sequence shown in SEQ ID NO: 22, which is obtained by performing substitution and/or deletion and/or addition of one or more amino acid residues on the amino acid sequence shown in SEQ ID NO: 22 in the sequence listing;

the mcr C-terminal domain can be the following a25) or a26):

a25) a protein having the amino acid sequence shown in SEQ ID NO: 23 in the sequence listing;

a26) a protein having 75% or more identity with and the same function as the amino acid sequence shown in SEQ ID NO: 23, which is obtained by performing substitution and/or deletion and/or addition of one or more amino acid residues on the amino acid sequence shown in SEQ ID NO: 23 in the sequence listing.

The gene encoding the mcr protein can be composed of the gene encoding the mcr N-terminal domain and the gene encoding the mcr C-terminal domain, and the gene encoding the mcr N-terminal domain can be the following b25) or b26):

b25) a DNA molecule having the nucleotide sequence shown in positions 1-1_689 of SEQ ID NO: 21 in the sequence listing;

b26) a DNA molecule having 75% or more identity with and the same function as the nucleotide sequence defined in b25);

the gene encoding the mcr C-terminal domain can be the following b27) or b28):

b27) a DNA molecule having the nucleotide sequence shown in positions 1704-3749 of SEQ ID NO: 21 in the sequence listing;

b28) a DNA molecule having 75% or more identity with and the same function as the nucleotide sequence defined in b27).

Still further, the gene encoding the mcr protein can be the following b29) or b30):

b29) a DNA molecule having the nucleotide sequence shown in SEQ ID NO: 21 in the sequence listing;

b30) a DNA molecule having 75% or more identity with and the same function as the nucleotide sequence defined in b29).

The sequence shown in positions 1-1689 of SEQ ID NO: 21 is the nucleotide sequence of the mcr N-terminal domain, the sequence shown in positions 1704-3749 is the nucleotide sequence of the mcr C-terminal domain, and the sequence shown in positions 1691-1696 is the sequence of the RBS site.

In the above method, the step of “introducing into the biological cell strain a gene encoding a mcr protein” can specifically be “introducing an expression vector containing the gene encoding the mcr protein into the biological cell strain”.

The expression vector can be a plasmid, a cosmid, a phage or virus vector. The plasmid can specifically be pYB1k or pLB1a, the sequence of the plasmid pYB1k is shown in SEQ ID NO: 6 in the sequence listing, and the sequence of the plasmid pLB1a is shown in SEQ ID NO: 24 in the sequence listing.

The expression vector containing the gene encoding the mcr protein can be pLB1a-mcr; the vector pLB1a-mcr is a recombinant vector obtained by inserting the gene encoding the mcr protein into the plasmid pLB1a, and can express the mcr protein.

In practical applications, it can be further determined whether the synthesis of branched-chain α-ketoacids needs to be inhibited according to whether the participation of branched-chain α-ketoacids is required in the production process of the target product; if the participation of branched-chain α-ketoacids is required in the production process of the target product, the synthesis of branched-chain α-ketoacids cannot be inhibited; if the participation of branched-chain α-ketoacids is not required in the production process of the target product, the synthesis of branched-chain α-ketoacids can be inhibited so as to further improve the yield of the target product.

In the above method, the target product can be picric acid or an intermediate product from malonyl-CoA to picric acid in the picric acid synthesis pathway, and the method comprises the steps of: introducing into the recombinant cell strain A a gene encoding a vps protein (phenylpentanone synthase) and expressing the encoding gene, or increasing the content of the vps protein or enhancing the activity of the vps protein in the recombinant cell strain A, to obtain a recombinant cell strain, which is recorded as recombinant cell strain-vps; culturing the recombinant cell strain-vps to prepare the target product.

Further, the vps protein and its encoding gene can be derived from Humulus lupulus;

still further, the vps protein can be the following a27) or a28):

a27) a protein having the amino acid sequence shown in SEQ ID NO: 26 in the sequence listing;

a28) a protein having 75% or more identity with and the same function as the amino acid sequence shown in SEQ ID NO: 26, which is obtained by performing substitution and/or deletion and/or addition of one or more amino acid residues on the amino acid sequence shown in SEQ ID NO: 26 in the sequence listing.

The gene encoding the vps protein can be the following b31) or b32):

b31) a DNA molecule having the nucleotide sequence shown in SEQ ID NO: 25 in the sequence listing;

b32) a DNA molecule having 75% or more identity with and the same function as the nucleotide sequence defined in b31).

In the above method, the step of “introducing into the biological cell strain a gene encoding a vps protein” can specifically be “introducing an expression vector containing the gene encoding the vps protein into the biological cell strain”.

The expression vector can be a plasmid, a cosmid, a phage or virus vector. The plasmid can specifically be pYB1k or pLB1a, the sequence of the plasmid pYB1k is shown in SEQ ID NO: 6 in the sequence listing, and the sequence of the plasmid pLB1a is shown in SEQ ID NO: 24 in the sequence listing.

The expression vector containing the gene encoding the vps protein can be pLB1a-vps: the vector pLB1a-vps is a recombinant vector obtained by inserting the gene encoding the vps protein into the plasmid pLB1a, and can express the vps protein.

The intermediate product does not include malonyl-CoA or picric acid. In one embodiment of the present invention, the intermediate product is 3-methyl-isobutyrylphloroglucinol (PIVP).

The present invention also provides a reagent set, the reagent set is reagent set A or reagent set B or reagent set C;

the reagent set A includes the branched-chain α-ketoacid dehydrogenase complex or the gene encoding the branched-chain α-ketoacid dehydrogenase complex;

the reagent set B consists of the reagent set A and the mcr protein or the gene encoding the mcr protein;

the reagent set C consists of the reagent set A and the vps protein or the gene encoding the vps protein.

The reagent set A can further include the ppc protein or the gene encoding the ppc protein.

The reagent set A can also include a substance that inhibits the synthesis of branched-chain α-ketoacids.

The substance that inhibits the synthesis of branched-chain α-ketoacids can be a substance required to knock out at least one gene in the branched-chain α-ketoacid synthesis pathway in the biological cell strain, or reduce the content or the activity of at least one protein encoded by the genes in the branched-chain α-ketoacid synthesis pathway.

The substance that inhibits the synthesis of branched-chain α-ketoacids can be the substance required to knock out the ilvA gene or/and the ilvE gene in the biological cell strain.

The biological cell strain contains the ilvA gene or/and the ilvE gene.

Specifically, the step of “knocking out the ilvA gene in the biological cell strain” can be using a gene fragment or a strain (e.g., Escherichia coli JW3745 strain) with the ilvA gene knockout trait.

Specifically, the step of “knocking out the ilvE gene in the biological cell strain” can be using a gene fragment or a strain (e.g., Escherichia coli JW5606 strain) with the ilvE gene knockout trait.

The reagent set A can only consist of the branched-chain α-ketoacid dehydrogenase complex or the gene encoding the branched-chain α-ketoacid dehydrogenase complex, and can also consist of the branched-chain α-ketoacid dehydrogenase complex or the gene encoding the branched-chain α-ketoacid dehydrogenase complex, and the ppc protein or the gene encoding the ppc protein, and can also consist of the branched-chain α-ketoacid dehydrogenase complex or the gene encoding the branched-chain α-ketoacid dehydrogenase complex, and the ppc protein or the gene encoding the ppc protein as well as the substance that inhibits the synthesis of branched-chain α-ketoacids.

The reagent set A has the following D1) or D2) use:

D1) synthesis of malonyl-CoA;

D2) production of a target product with malonyl-CoA as an intermediate product.

The reagent set B can be used to produce 3-hydroxypropionic acid.

The reagent set C can be used to prepare picric acid or an intermediate product from malonyl-CoA to picric acid in the picric acid synthesis pathway.

The present invention also provides a recombinant cell strain, which is the recombinant cell strain A, the recombinant cell strain-mcr or the recombinant cell strain-vps.

The present invention also provides the following I, II or III use:

I. use of the branched-chain α-ketoacid dehydrogenase complex or the gene encoding the branched-chain α-ketoacid dehydrogenase complex, the recombinant cell strain A or the reagent set A in any one of the followings:

X1) synthesis of malonyl-CoA;

X2) preparation of a product for the synthesis of malonyl-CoA;

X3) production of a target product with malonyl-CoA as an intermediate product;

X4) preparation of a product for the production of a target product with malonyl-CoA as an intermediate product;

X5) synthesis of 3-hydroxypropionic acid;

X6) preparation of a product for the synthesis of 3-hydroxypropionic acid;

X7) synthesis of picric acid or an intermediate product from malonyl-CoA to picric acid in the picric acid synthesis pathway;

X8) preparation of a product for the synthesis of picric acid or an intermediate product from malonyl-CoA to picric acid in the picric acid synthesis pathway;

X9) synthesis of fatty acids;

X10) preparation of a product for the synthesis of fatty acids;

X11) synthesis of polyketides;

X12) preparation of a product for the synthesis of polyketides;

X13) synthesis of flavonoids;

X14) preparation of a product for the synthesis of flavonoids;

II. use of the recombinant cell strain-mcr or the reagent set B in any one of the followings:

Y1) synthesis of 3-hydroxypropionic acid;

Y2) preparation of a product for the synthesis of 3-hydroxypropionic acid;

III. use of the recombinant cell strain-vps or the reagent set C in any one of the followings:

Z1) synthesis of picric acid or an intermediate product from malonyl-CoA to picric acid in the picric acid synthesis pathway;

Z2) preparation of a product for the synthesis of picric acid or an intermediate product from malonyl-CoA to picric acid in the picric acid synthesis pathway.

The synthesis of malonyl-CoA uses oxaloacetate as a substrate.

In the synthetic pathway of the target product, the participation of malonyl-CoA is required.

The target product can be 3-hydroxypropionic acid, picric acid or an intermediate product from malonyl-CoA to picric acid in the picric acid synthesis pathway.

The intermediate product does not include malonyl-CoA or picric acid. In one embodiment of the present invention, the intermediate product is 3-methyl-isobutyrylphloroglucinol (PIVP).

In the present invention, the phrase of “having 75% or more identity” refers to having 75%, 80%, 85%, 90%, 95%, 96%, 97%, 98%, or 99% identity.

### DESCRIPTION OF THE DRAWINGS

FIG. 1 shows the detection results of the relative contents of malonyl-CoA of the engineered strains expressing the branched-chain α-ketoacid dehydrogenase complex.

FIG. 2 shows the detection results of the relative content of malonyl-CoA after introducing the ppc gene into M-FGH.

FIG. 3 shows the yields of 3-hydroxypropionic acid after expressing the branched-chain α-ketoacid dehydrogenase complex.

FIG. 4 shows the α/β acid metabolic pathway in Humulus lupulus.

FIG. 5 shows the yields of PIVP of the engineered strains after expressing the branched-chain α-ketoacid dehydrogenase complex.

FIG. 6 is the in vitro enzyme activity assay of branched-chain α-ketoacid dehydrogenase complex. Oxaloacetate is the OAA group, oxaloacetate-EDTA is the OAA-EDTA group, 3-methyl-2-oxobutanoic acid is the KIV group, and 3-methyl-2-oxobutanoic acid-EDTA is the KIV-EDTA group.

### DETAILED DESCRIPTION OF THE INVENTION

The present invention will be further described in detail below with reference to the specific embodiments, and the given examples are only for illustrating the present invention, rather than for limiting the scope of the present invention. The experimental methods in the following examples are conventional methods unless otherwise specified. Materials, reagents, instruments, etc. used in the following examples are all commercially available unless otherwise specified. The quantitative tests in the following examples were performed in triplicate, and the results were averaged. In the following examples, unless otherwise specified, the first position of each nucleotide sequence in the sequence listing is the 5′-end nucleotide of the corresponding DNA/RNA, and the last position is the 3′-end nucleotide of the corresponding DNA/RNA.

In the following examples, Escherichia coli BW25113 (Datsenko K A, Wanner B L. One-step inactivation of chromosomal genes in Escherichia coli K-12 using PCR products. Proc. Natl. Acad. Sci. U.S.A. 2000; 97(12): 6640-6645.) is a non-pathogenic bacterial strain with clear genetic background, short generation time, easy cultivation and low-cost medium raw materials. This bacterial strain contains oxaloacetate synthesis pathway and can synthesize oxaloacetate. Escherichia coli BW25113 is available to the public from the Institute of Microbiology, Chinese Academy of Sciences. This biological material is only used for repeating the relevant experiments of the present invention, and cannot be used for other purposes.

The wild-type P1 phage in the following examples (Thomason L C, Costantino N. 2007. E. coli genome manipulation by P1 transduction. Current Protocols in Molecular Biology: 1.17. 1-8) is available to the public from the Institute of Microbiology, Chinese Academy of Sciences. This biological material is only used for repeating the relevant experiments of the present invention, and cannot be used for other purposes.

Example 1. Branched-chain α-ketoacid dehydrogenase complex can catalyze the synthesis of malonyl-CoA

It is found in the present invention that the branched-chain α-ketoacid dehydrogenase complex can catalyze the synthesis of malonyl-CoA. In this example, the genes encoding the branched-chain α-ketoacid dehydrogenase complex (bkdA, bkdB, bkdC, lpdA1, bkdF, bkdG, bkdH genes) were prepared, and two genes in the branched-chain α-ketoacid synthesis pathway (threonine deaminase ilvA gene and branched-chain amino acid transaminase ilvE gene) were knocked out. The synthesis of malonyl-CoA catalyzed by the α-ketoacid dehydrogenase complex was detected, and the primers used are shown in Table 1.

(1) Construction of plasmids expressing the branched-chain α-ketoacid dehydrogenase complex of Streptomyces avermitilis

(1-a) PCR amplification of bkdA, bkdB, bkdC, lpdA1, bkdF, bkdG and bkdH genes

A bacterial genome extraction kit (Tiangen Biotech Co., Ltd., item number: DP302) was used to extract the genomic DNA of Streptomyces avermitilis. Using the extracted Streptomyces avermitilis genomic DNA as a template, PCR was performed with primers bkdA-NcoI and bkdC-rbs-R using high-fidelity TransStart FastPfu DNA polymerase (TransGen Biotech, item number: AP221) and the obtained gene fragment was recorded as ABC, which contains the DNA fragment shown in SEQ ID NO: 1 in the sequence listing; PCR was performed with primers bkdF-NcoI and bkdH-rbs-R and the obtained gene fragment was recorded as FGH, which contains the DNA fragment shown in SEQ ID NO: 2 in the sequence listing; PCR was performed with primers bkdF-NcoI and bkdG-rbs-R and the obtained gene fragment was recorded as FG, which contains the sequence shown in positions 1-2200 of SEQ ID NO: 2 in the sequence listing: PCR was performed with primers rbs-lpdA1-F and lpdA1-XhoI, and the obtained gene fragment was recorded as lpd, which contains the lpdA1 gene shown in SEQ ID NO: 4 in the sequence listing.

Among them, the sequence shown in positions 1-1146 of SEQ ID NO: 1 is the DNA sequence of bkdA gene, which encodes the bkdA protein shown in SEQ ID NO: 7 in the sequence listing; the sequence shown in positions 1220-2224 is the DNA sequence of bkdB gene, which encodes the bkdB protein shown in SEQ ID NO: 8 in the sequence listing; the sequence shown in positions 2224-3591 is the DNA sequence of bkdC gene, which encodes the bkdC protein shown in SEQ ID NO: 9 in the sequence listing;

the sequence shown in positions 1-1221 of SEQ ID NO: 2 is the DNA sequence of bkdF gene, which encodes the bkdF protein shown in SEQ ID NO: 10 in the sequence listing; the sequence shown in positions 1223-2200 of SEQ ID NO: 2 is the DNA sequence of bkdG gene, which encodes the bkdG protein shown in SEQ ID NO: 11 in the sequence listing; the sequence shown in positions 2220-3608 of SEQ ID NO: 2 is the DNA sequence of bkdH gene, which encodes the bkdH protein shown in SEQ ID NO: 12 in the sequence listing;

The lpdA1 gene shown in SEQ ID NO: 4 encodes the lpdA1 protein shown in SEQ ID NO: 13 in the sequence listing.

According to the codon preference in Escherichia coli, the sequences of bkdH and lpdA1 genes were optimized, respectively, and the optimized genes were recorded as opbkdH and oplpdA1 genes, respectively. The sequences of opbkdH and oplpdA1 genes were shown in SEQ ID NO: 3 and SEQ ID NO: 5 in the sequence listing, respectively. SEQ ID NO: 3 and SEQ ID NO: 5 encode the bkdH protein and lpdA1 protein shown in SEQ ID NOs: 12 and 13 in the sequence listing, respectively. The opbkdH and oplpdA1 genes were artificially synthesized; using the opbkdH gene as a template, PCR was performed with rbs-opbkdH-F and rbs-opbkdH-R and the obtained gene fragment was recorded as opH, which contains the opbkdH gene shown in SEQ ID NO: 3; using the oplpdA1 gene as a template, PCR was performed with rbs-oplpdA1-F and oplpdA1-XhoI, and the obtained gene fragment was recorded as oplpd, which contains the oplpdA1 gene shown in SEQ ID NO: 5 in the sequence listing.

(1-b) Construction of recombinant expression vectors containing bkdA, bkdB, bkdC, lpdA1, bkdF, bkdG and bkdH genes

Each PCR amplified fragment obtained in the above step (1-a) was analyzed by agarose gel electrophoresis and the target fragment was recovered; at the same time, the vector pYB1k (the nucleotide sequence of the vector pYB1k is shown in SEQ ID NO: 6 in the sequence listing) was digested with NcoI and XhoI enzymes and the large vector fragment YB1k-NX fragment (i.e., the vector backbone) was recovered. Using the Gibson assembly method (Gibson D G, Young L, et al. Enzymatic assembly of DNA molecules up to several hundred kilobases. Nat, methods. 2009; 6(5): 343-345), the recovered ABC and lpd fragments were ligated with the YB1k-NX fragment; the recovered FGH, lpd fragments and the YB1k-NX fragment were subjected to Gibson assembly ligation reaction; the recovered FG, opH, oplpd and YB1k-NX fragment were subjected to Gibson assembly ligation reaction. Each ligation product was transformed into Escherichia coli DH5α competent cells (TransGen Biotech, item number: CD201) by the CaCl₂ method, then spread evenly on a LB plate containing kanamycin, and cultured at 37° C. overnight. Clones were picked and the clones from which the target fragment could be amplified with primers F108/R124 were identified and sequenced; positive clones were picked to extract plasmids, and the correct recombinant plasmid obtained by ligating the ABC, lpd fragments with the YB1k-NX fragment was named pYB1k-bkdABC-lpdA1, the correct recombinant plasmid obtained by ligating the FGH, lpd fragments with the YB1k-NX fragment was named pYB1k-bkdFGH-lpdA1, and the correct recombinant plasmid obtained by ligating the FG, opH, oplpd fragments with the YB1k-NX fragment was named pYB1k-bkdFG-opbkdH-oplpdA 1.

pYB1k-bkdABC-lpdA1 contains the DNA fragments shown in SEQ ID NOs: 1 and 4 in the sequence listing and can express the four proteins shown in SEQ ID NOs: 7, 8, 9 and 13; pYB1k-bkdFGH-lpdA1 contains the DNA fragments shown in SEQ ID NOs: 2 and 4 in the sequence listing and can express the four proteins shown in SEQ ID NOs: 10, 11, 12 and 13; pYB1k-bkdFG-opbkdH-oplpdA1 contains the DNA fragments shown in positions 1-2200 of SEQ ID NO: 2, SEQ ID NO: 3 and SEQ ID NO: 4 in the sequence listing and can express the four proteins shown in SEQ ID NOs: 10, 11, 12 and 13.

(2) Knockout of threonine deaminase ilvA gene and branched-chain amino acid transaminase ilvE gene in the engineered strain

Using Escherichia coli BW251113 as the starting strain, the ilvA gene was knocked out, and the obtained recombinant strain was recorded as M01A, and the ilvE gene was knocked out, and the obtained recombinant strain was recorded as M01E.

(2-a) Preparation of P1 phages containing E. coli gene fragments with ilvA and ilvE gene knockout traits

The E. coli gene fragment with the ilvA gene knockout trait and the E. coli gene fragment with the ilvE gene knockout trait are derived from E. coli strains JW3745 and JW5606, respectively, which are W3110 strains containing the ilvA and ilvE knockout traits, respectively. Both the two strains are from the NIG, Japan, and the ilvA gene encoding threonine deaminase and the ilvE gene encoding branched-chain amino acid transaminase in these two strains were both replaced with a kanamycin resistance gene with FRT sites at both ends (about 1300 bp) to knock out the ilvA or ilvE gene (Baba T, Ara T, et al. Construction of Escherichia coli K-12 in-frame, single-gene knockout mutants: the Keio collection. Mol. Syst. Biol. 2006; 2:2006. 0008.). The preparation of P1 phage is as follows: the JW3745 or JW5606 strain was cultured at 37° C. overnight, then transferred to LB medium containing 5 mmol/L CaCl₂ and 0.1% glucose; the cultivation was continued at 37° C. for 1 h, and then added with wild-type P1 phage to continue the cultivation for 1-3 h; a few drops of chloroform were added and the cultivation was continued for several minutes; the culture solution was centrifuged to obtain the supernatant, i.e., phage P1vir ilvA containing the E. coli gene fragment with the ilvA gene knockout trait and phage P1vir ilvE containing the E. coli gene fragment with the ilvE gene knockout trait.

(2-b) Construction of E. coli strains M01A-Kan and M01E-Kan using P1 phage transduction technology

For overnight cultured Escherichia coli BW25113 (recipient bacteria), 1.5 mL of the bacterial solution was centrifuged at 10,000 g for 2 min, and then the BW25113 cells were resuspended with 0.75 mL of P1 salt solution (the solvent was water and the solutes were 10 mM CaCl₂ and 5 mM MgSO₄). One hundred μL of phage P1vir ilvA or P1vir ilvE was mixed with 100 μL of BW25113 cell suspension, incubated at 37° C. for 30 min. One mL of LB medium and 200 μL of 1 mol/L sodium citrate were added, and the incubation was continued at 37° C. for 1 h. The cells were collected by centrifugation, resuspended in 100 μL of LB medium, and spread on a LB plate containing kanamycin (the concentration of kanamycin was 50 μg/ml). The plate was incubated overnight at 37° C. Clones were picked and identified by PCR with primers ilvA-F/ilvA-R or ilvE-F/ilvE-R (a 1700 bp target band was amplified from positive clones), and positive clones were screened. The positive clone obtained from phage P1vir ilvA was named MW01A-Kan, the positive clone obtained from phage P1vir ilvE was named MW01E-Kan.

(2-c) Elimination of resistance

The plasmid pCP20 (Clontech company) was transformed into M01A-Kan and M01E-Kan by the calcium chloride transformation method, and clones were picked after overnight cultivation on a LB plate containing ampicillin at 30° C. to obtain recombinant E. coli strains containing plasmid pCP20, i.e., M01A-Kan/pCP20 and M01E-Kan/pCP20. These two strains were cultured in LB medium containing ampicillin at 30° C., respectively, spread on LB plates without antibiotic and cultured overnight at 42° C. Clones were picked and identified by PCR with primers ilvA-F/ilvA-R or ilvE-F/ilvE-R (a 400 bp target band was amplified from positive clones), and positive clones were screened. The positive clone obtained from M01A-Kan was named M01A, i.e., the ilvA gene knockout strain of E. coli BW25113, and the positive clone obtained from M01E-Kan was named M01E, i.e., the ilvE gene knockout strain of E. coli BW25113.

In Escherichia coli BW25113, the coding sequence of the ilvA gene is shown in SEQ ID NO: 14 in the sequence listing, which encodes the ilvA protein shown in SEQ ID NO: 15 in the sequence listing; the coding sequence of the ilvE gene is shown in SEQ ID NO: 16 in the sequence listing, which encodes the ilvE protein shown in SEQ ID NO: 1-7 in the sequence listing.

TABLE 1
List of primers used

| | | Used |
|---|---|---|
| Primer | Sequence | in |
| bkdA-NcoI | 5′-TAACAGGAGGAATTAACCAT | Step |
| | GACGGTCATGGAGCAGCGG-3′ | (1) |
| | (SEQ ID NO: 29) | |
| bkdC-rbs-R | 5′-CGGTGCTGGCGTCGTTCGCC | Step |
| | ATTTAATTCCTCCTGACTAC | (1) |
| | AGGGTGCGCAGCAGCACCGC | |
| | CGG-3′ | |
| | (SEQ ID NO: 30) | |
| bkdF-NcoI | 5′-GCTAACAGGAGGAATTAACC | Step |
| | GTGACCGTGGAGAGCACTGC | (1) |
| | CGC-3′ | |
| | (SEQ ID NO: 31) | |
| bkdH-rbs-R | 5′-CGGTGCTGGCGTCGTTCGCC | Step |
| | ATTTAATTCCTCCTGACTAG | (1) |
| | GCCCAGGTGATCAGCCGCTT | |
| | CGGC | |
| | -3′ | |
| | (SEQ ID NO: 32) | |
| bkdG-rbs-R | 5′-TTAATTCCTCCTGATCAGTA | Step |
| | CGCCAGCGAGCGGTCGACGG | (1) |
| | CATCG-3′ | |
| | (SEQ ID NO: 33) | |
| rbs-lpdA1-F | 5′-TCAGGAGGAATTAAATGGCG | Step |
| | AACGACGCCAGCACCG-3′ | (1) |
| | (SEQ ID NO: 34) | |
| lpdA1-XhoI | 5′-TAGTACCAGATCTACCCTCG | Steps |
| | AGTCAGTCGTGCGAGTGCAG | (1 |
| | CGGCTTGCCCGCGAGGG-3′ | and |
| | (SEQ ID NO: 35) | 10) |
| rbs-opbkdH-F | 5′-TCGCTGGCGTACTGATCAGG | Step |
| | AGGAATTAAATGACTGAAGC | (1) |
| | GTCCGTGCGTG-3′ | |
| | (SEQ ID NO: 36) | |
| rbs-opbkdH-R | 5′-TTAATTCCTCCTGATTATGC | Step |
| | CCAAGTGATGAGACGCTTCG | (1) |
| | GCT-3′ | |
| | (SEQ ID NO: 37) | |
| rbs-oplpdA1-F | 5′-CTCATCACTTGGGCATAATC | Step |
| | AGGAGGAATTAAATGGCAAA | (1) |
| | CGACGCATCTACG-3′ | |
| | (SEQ ID NO: | |
| | 38) | |
| oplpdA1-XhoI | 5′-TAGTACCAGATCTACCCTCG | Step |
| | AGTTAGTCGTGAGAATGCAG | (1) |
| | CGGCTTGCCAGCCAG-3′ | |
| | (SEQ ID NO: 39) | |
| F108 | 5′-CGGCGTCACACTTTGCTAT | Steps |
| | G-3′ | (1, 8 |
| | (SEQ ID NO: 40) | and |
| | | 10) |
| R124 | 5′-CGTTTCACTTCTGAGTTCGG | Step |
| | C-3′ | (1) |
| | (SEQ ID NO: 41) | |
| ilvA-F | 5′-GATTGAAGATGGTGACCTGA | Step |
| | TCGCTATC-3′ | (2) |
| | (SEQ ID NO: 42) | |
| ilvA-R | 5′-GCTGTGGCGTGGCATTGTTG | Step |
| | CCGGAAG-3′ | (2) |
| | (SEQ ID NO: 43) | |
| ilvE-F | 5′-TGTATCGGCTCGCTTCAATC | Step |
| | CAGAAACCT-3′ | (2) |
| | (SEQ ID NO: 44) | |
| ilvE-R | 5′-AATTTGTTCGGCGACCAGTT | Step |
| | TACCGAGA-3′ | (2) |
| | (SEQ ID NO: 45) | |

(3) Exogenous expression of branched-chain α-ketoacid dehydrogenase complex improves the synthesis of malonyl-CoA in engineered strains

(3-a) Preparation of recombinant strains

The recombinant vectors pYB1k-bkdABC-lpdA1, pYB1k-bkdFGH-lpdA1 and pYB1k-bkdFG-opbkdH-oplpdA1 of step (1) and the vector pYB1k were introduced into Escherichia coli BW25113, respectively, to obtain recombinant strains, which were recorded as M-ABC, M-FGH, M-opFGH and BW, respectively; the recombinant vector pYB1k-bkdFGH-lpdA1 was introduced into M01 A and M01E of step (1), respectively, to obtain recombinant strains, which were recorded as MA-FGH and ME-FGH, respectively.

(3-b) Preparation of Culture Medium

Medium A: a sterile medium composed of a solvent and solutes; solvent: water; solutes and their concentrations in the medium: NaHPO₄ 25 mM, KH₂PO₄ 25 mM, NH₄Cl 50 mM.

Medium B: a sterile medium obtained by adding Na₂SO₄, MgSO₄, glycerol, yeast powder and trace elements to medium A; in medium B, Na₂SO₄ concentration: 5 mM, MgSO₄ concentration: 2M, glycerol volume percentage: 0.5%, yeast powder mass percentage: 0.5 mg/100 mL, and each trace element and its concentration: 50 μM FeCl₃, 20 μM CaCl₂, 10 μM MnCl₂, 10 μM ZnSO₄, 2 μM CoCl₂, 2 μM NiCl₂, 2 μM Na₂MO₄, 2 μM Na₂SeO₃ and 2 μM H₃BO₃.

Medium C: a sterile medium obtained by adding glucose to medium A; glucose concentration in medium C: 20 g/L.

(3-c) Culture of Cells and Induction of Enzymes

The overnight cultured engineered strain M-ABC was inoculated into 20 ml of medium B in a shake flask at an inoculum concentration of 1%; after culturing at 37° C. for 6 h, arabinose was added to the culture system to a final mass percentage of 0.2%; the cultivation was continued for 12 h, and the cells (i.e., M-ABC cells) were collected.

According to the above method, the strain M-ABC was replaced with M-FGH, M-opFGH, BW, MA-FGH and ME-FGH, respectively, to obtain M-FGH, M-opFGH, BW, MA-FGH and ME-FGH cells.

(3-d) Whole-Cell Catalysis of Malonyl-CoA

The same amount of cells collected in step (3-c) were taken (the amount of the cells used: 1 mL of cell suspension with an OD600 of 90) to detect the synthetic amount of malonyl-CoA in each strain according to the following method:

the cells were resuspended in 5 ml of medium C in a shake flask; after culturing at 37° C. for 3 h, the cells were collected by centrifugation, and then the cells were resuspended in 400 μL of 80% (volume percent) methanol aqueous solution pre-cooled at −80° C.; the cells were disrupted by sonication, centrifuged at 12,000 rpm at 4° C. for 20 min, and the supernatant was collected to detect the content of malonyl-CoA in the supernatant. The content of malonyl-CoA in the supernatant was analyzed by LCMS/MS using the standard curve method (external standard method) with malonyl-CoA (Sigma, 63410-10MG-F) as the standard.

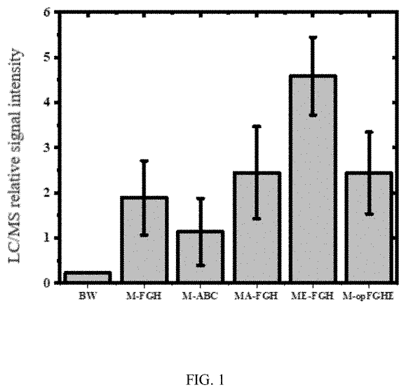

The results are shown in FIG. 1. In the figure, the ordinate is the detected relative signal intensity of malonyl-CoA, and the relative signal intensities of malonyl-CoA in the supernatants of B W, M-ABC, M-FGH, M-opFGH, MA-FGH and ME-FGH were 0.22, 1.13, 1.89, 2.43, 2.44 and 4.59, respectively. Compared with BW, the relative contents of malonyl-CoA in the supernatants of M-ABC, M-FGH, M-opFGH, MA-FGH and ME-FGH were 5.09, 8.48, 10.94, 10.98 and 20.61, respectively, that is, the contents of malonyl-CoA in the supernatants of M-ABC, M-FGH, M-opFGH, MA-FGH and ME-FGH were 5.09 times, 8.48 times, 10.94 times, 10.98 times, and 20.61 times that of BW, respectively.

The results showed that the synthesis of malonyl-CoA was significantly increased after the gene encoding branched-chain α-ketoacid dehydrogenase complex was introduced into Escherichia coli; compared with the introduction of bkdA, bkdB, bkdC and lpdA1 genes, the introduction of bkdF, bkdG, bkdH and lpdA1 genes results in a higher synthetic amount of malonyl-CoA; while bkdH and lpdA1 genes were optimized according to the codon preference of E. coli, the synthetic amount of malonyl-CoA could be further improved; based on the introduction of bkdF, bkdG, bkdH and lpdA1 genes, the knockout of the ilvA and ilvE genes in the branched-chain α-ketoacid (the substrate of the branched-chain i-ketoacid dehydrogenase complex) synthesis pathway, the synthetic amount of malonyl-CoA could be further improved, and the synthetic amount of malonyl-CoA could be greatly increased after the knockout of the ilvE gene.

Example 2. Phosphoenolpyruvate carboxylase ppc gene can improve the synthetic amount of malonyl-CoA in the engineered strain M-FGH

In this example, on the basis of the engineered strain M-FGH of Example 1, phosphoenolpyruvate carboxylase ppc gene was introduced and ompT gene was knocked out (the sequence of the ompT gene is shown in SEQ ID NO: 27 in the sequence listing, which encodes the ompT protein shown in SEQ ID NO: 28) and the synthetic amount of malonyl-CoA was further improved. The primers used are shown in Table 2.

(4) Construction of an engineered strain expressing phosphoenolpyruvate carboxylase ppc gene

(4-a) Extraction of Corynebacterium glutamicum and Escherichia coli genomes, and PCR amplification of ppc gene, chloramphenicol resistance fragment and upstream and downstream homologous arms of ompT gene

ppc-F and ppc-R were used as PCR amplification primers, and the genomic DNA of Corynebacterium glutamicum was used as the template to amplify the fragment tac-ppc. The fragment tac-ppc contains the ppc gene, and the ppc gene has a nucleotide sequence shown in SEQ ID NO: 18 in the sequence listing and encodes the ppc protein shown in SEQ ID NO: 19. Cm-F and Cm-R were used as PCR amplification primers, and lox71-Cm-lox66-tac was used as the template to amplify the fragment Cm. The fragment lox71-Cm-lox66-tac has a nucleotide sequence shown in SEQ ID NO: 20 in the sequence listing. This fragment was obtained by whole gene synthesis (GenScript, Nanjing). ompT-up-F and ompT-up-R were used as PCR amplification primers, and the genomic DNA of Escherichia coli was used as the template to amplify the fragment ompT-up. ompT-down-F and ompT-down-R were used as PCR amplification primers and the genomic DNA of Escherichia coli was used as the template to amplify the fragment ompT-down.

(4-b) Preparation of Targeting Fragment ompT-Up-Cm-Tac-Ppc-ompT-Down

The four fragments tac-ppc, Cm, ompT-up and ompT-down were used as the template, and ompT-up-F and ompT-down-R were used as primers to obtain the targeting fragment ompT-up-Cm-tac-ppc-ompT-down by fusion PCR. The targeting fragment was recovered by agarose gel electrophoresis (Tiangen Biotech Co., L td., item number: DP209).

(4-c) Preparation of Host Strain Containing Plasmid pKD46

The plasmid pKD46 (Clontech company) was transformed into the engineered strain M-FGH by the calcium chloride transformation method, and clones were picked after overnight cultivation on a LB plate containing ampicillin and kanamycin at 30° C. to obtain the recombinant E. coli strain M-FGH/pKD46 containing plasmid pKD46. After the recombinant E. coli strain M-FGH/pKD46 was induced by arabinose, three recombinant proteins of the phage were expressed, and the host strain acquired the ability of homologous recombination. M-FGH/pKD46 competent cells were then prepared by washing with 10% glycerol.

(4-d) Homologous Recombination

The fragment ompT-up-Cm-tac-ppc-ompT-down of step (4-b) was electro-transformed into the M-FGH/pKD46 competent cells prepared in step (4-c), and the cells were cultured on a LB plate containing kanamycin (concentration: 50 μg/ml) and chloramphenicol (concentration: 34 μg/ml) overnight at 37° C. Clones were picked and identified by PCR with ompT-up1k-F and ppc-R primers (a 6000 bp target band was amplified from positive clones), a positive clone was screened and named M-FGH-ppc. M-FGH-ppc contains the ppc gene shown in SEQ ID NO: 18 in the sequence listing, and can express the ppc protein shown in SEQ ID NO: 19. M-FGH-ppc does not contain the ompT gene.

(5) Detection of synthetic amount of malonyl-CoA in the engineered strain overexpressing phosphoenolpyruvate carboxylase ppc gene and Streptomyces avermitilis branched-chain α-ketoacid dehydrogenase complex gene bkdFGH-lpdA1

According to the methods of (3-c) and (3-d) of step (3) in Example 1, M-ABC was replaced by M-FGH and M-FGH-ppc, respectively, and other steps were unchanged. The synthetic amounts of malonyl-CoA in the strains were detected,

The results are shown in FIG. 2. In the figure, the ordinate is the detected relative signal intensity of malonyl-CoA, and the relative signal intensities of malonyl-CoA in the supernatants of M-FGH and M-FGH-ppc were 1.89 and 3.66, respectively. Compared with M-FGH, the relative content of malonyl-CoA in the supernatant of M-FGH-ppc was 1.94, that is, the content of malonyl-CoA in the supernatant of M-FGH-ppc was 1.94 times that of M-FGH. The results showed that ppc gene could increase the synthetic amount of malonyl-CoA.

TABLE 2
List of primers

| | | Used |
|---|---|---|
| Primer | Sequence | in |
| ppc-F | 5′-ATGACTGATTTTTTACGCG | Step |
| | ATGACATCA-3′ | (4) |
| | (SEQ ID NO: 46) | |
| ppc-R | 5′-CCCCGGGGCGATTTTCACC | Step |
| | TCGGGGAAATTTTAGTTGGCGTTC | (4) |
| | CTAGCCGGAGTTGCGCAGCGCAG-3′ | |
| | (SEQ ID NO: 47) | |
| Cm-F | 5′-ACATATTCAATCATTAAAA | Step |
| | CGATTGAATGGAGAACTTTTGTCT | (4) |
| | CGAGAATATCCTCCTTATAACTT-3′ | |
| | (SEQ ID NO: 48) | |
| Cm-R | 5′-GATTTGACCGAGGAACCTG | Step |
| | ATGTCATCGCGTAAAAAATCAGTC | (4) |
| | ATGGTTAATTCCTCCTTCCACAC-3′ | |
| | (SEQ ID NO: 49) | |
| ompT-up-F | 5′-ACTGGAATCTGCGAATTGT | Step |
| | CGCCAGT-3′ | (4) |
| | (SEQ ID NO: 50) | |
| ompT-up-R | 5′-AAAAGTTCTCCATTCAATCGT | Step |
| | TTTAA-3′(SEQ ID NO: 51) | (4) |
| ompT-down- | 5′-GAACGCCAACTAAAATTTCC | Step |
| F | CCGAG-3′ | (4) |
| | (SEQ ID NO: 52) | |
| ompT-down- | 5′-AAAAGTTCTCCATTCAATCG | Step |
| R | TTTTAA-3′ (SEQ ID NO: 53) | (4) |
| ompT-uplk- | 5′-CCGTACACCGGAAGTGTTCC | Step |
| F | GGCTA-3′(SEQ ID NO: 54)) | (4) |

Example 3. Expression of Streptomyces avermitilis branched-chain α-ketoacid dehydrogenase complex gene bkdFGH-lpdA1 can increase the yield of 3-hydroxypropionic acid (3-HP).

3-Hydroxypropionic acid is an important platform compound and a raw material for the synthesis of various chemicals. Malonyl-CoA can be used as a precursor to obtain 3-hydroxypropionic acid through a two-step reduction reaction. In this example, the malonyl-CoA reductase gene, mcr gene, of Chloroflexus aurantiacus was introduced into M-ABC, M-FGH, M-opFGH, MA-FGH, ME-FGH, and M-FGH-ppc obtained in Example 1 and Example 2 to prepare 3-hydroxypropionic acid, and BW of Example 1 was used as a control. The primers used are shown in Table 3.

(6) Construction of a plasmid expressing the malonyl-CoA reductase gene mcr of Chloroflexus aurantiacus

(6-a) The nucleotide sequence of the modified Chloroflexus aurantiacus malonyl-CoA reductase gene, mcr gene, is shown in SEQ ID NO: 21 in the sequence listing, wherein the nucleotide sequence of the N-terminal domain of the mcr gene is shown in positions 1-1689 of SEQ ID NO: 21 and encodes the N-terminal domain of mcr shown in SEQ ID NO: 22 in the sequence listing; the nucleotide sequence of the C-terminal domain of the mcr gene is shown in positions 1704-3749 of SEQ ID NO: 21 and encodes the C-terminal domain of mcr shown in SEQ ID NO: 23 in the sequence listing; a RBS site is contained between the N-terminal domain and the C-terminal domain, and has the sequence shown in positions 1691-1696 of SEQ ID NO: 21. The mcr gene fragment shown in SEQ ID NO: 21 was synthesized by whole gene synthesis and ligated to the vector pUC57 to obtain the recombinant vector pUC57-mcr. Using pUC57-mcr as the template, a PCR amplified fragment was obtained by amplifying with the primers mcr-F/mcrR.

(6-b) The PCR amplified fragment obtained in above step (6-a) was subjected to agarose gel electrophoresis to recover the target fragment; at the same time, the vector pLB1a (the nucleotide sequence of the vector pLB1a is shown in SEQ ID NO: 24 in the sequence listing) was digested with NcoI and XhoI, and the large vector fragment LB1a-NX (i.e., the vector backbone) was recovered. The above recovered target fragment was ligated with the LB1a-NX fragment by the Gibson assembly method, and the ligated product was transformed into Escherichia coli DH5α competent cells (TransGen Biotech, item number: CD201) by the CaCl₂ method. The cells were then spread on a LB plate containing streptomycin, and cultured at 37° C. overnight. Clones were picked and the clones from which the target fragment could be amplified with primers F-105/mcr-R were identified and sequenced. A positive clone was screened and the plasmid was extracted, and the obtained positive plasmid with the correct sequence was named pLB1a-mer.

pLB1a-mcr contains the mcr gene shown in SEQ ID NO: 21 in the sequence listing, and can express the N-terminal domain and the C-terminal domain of mcr shown in SEQ ID NOs: 22 and 23.

(7) Construction of a 3-hydroxypropionic acid-producing engineered strain and whole-cell catalysis of 3-hydroxypropionic acid

(7-a) The vector pLB1a-mcr obtained in step (6) was introduced into M-ABC, M-FGH, M-opFGH, MA-FGH, ME-FGH, M-FGH-ppc and BW respectively, to obtain recombinant strains, which were named M-ABC-HP, M-FGH-HP, M-opFGH-HP, MA-FGH-HP, ME-FGH-HP, M-FGH-ppc-HP and BW-HP. Each recombinant strain was used as the test strain for the production of 3-hydroxypropionic acid.

(7-b) Cultivation of Engineered Strains and Induction of Enzymes

Each overnight cultured test strain was inoculated into 20 ml of medium B described in (3-b) in a shake flask at an inoculum concentration of 1%; after culturing at 37° C. for 6 h, arabinose was added to the culture system to a final mass percentage of 0.2%; the cultivation was continued for 12 h, and the cells were collected.

(7-c) Whole-Cell Catalysis of 3-Hydroxypropionic Acid

The cells collected above were resuspended in 5 ml of medium C in a shake flask; after culturing at 37° C. for 8 h, the culture was centrifuged and the supernatant was obtained and filtered, and the filtrate was collected. The amount of cells used was 5 mL of cell suspension with an OD600 of 30. Using 3-hydroxypropionic acid (TCI, H0297-10 G) as the standard, the content of 3-hydroxypropionic acid in the filtrate was quantitatively analyzed by HPLC using the standard curve method (external standard method).

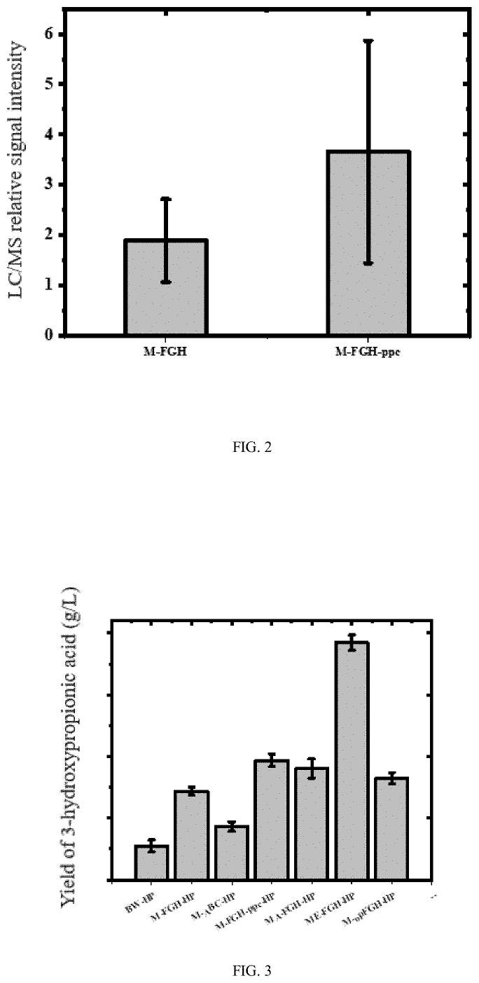

The results are shown in FIG. 3, the contents of 3-hydroxypropionic acid in the filtrates obtained from M-ABC-HP, M-FGH-HP, M-opFGH-HP, MA-FGH-HP, ME-FGH-HP, M-FGH-ppc-HP and BW-HP were 0.86, 1.44, 1.65, 1.80, 3.84, 1.94 and 0.55 g/L, respectively; the yields of 3-hydroxypropionic acid of M-ABC-HP, M-FGH-HP, M-opFGH-HP, MA-FGH-HP, ME-FGH-HP and M-FGH-ppc-HP were 1.56, 2.62, 3.00, 3.27, 6.98 and 3.53 times that of BW-HP, respectively.

The results showed that the yield of 3-hydroxypropionic acid was significantly increased after the gene encoding branched-chain α-ketoacid dehydrogenase complex was introduced into E. coli; compared with the introduction of bkdA, bkdB, bkdC and lpdA1 genes, the introduction of bkdF, bkdG, bkdH and lpdA1 genes results in a higher yield of 3-hydroxypropionic acid; while bkdH and lpdA1 genes were optimized according to the codon preference of E. coli, the yield of 3-hydroxypropionic acid could be further improved; based on the introduction of bkdF, bkdG, bkdH and lpdA1 genes, the knockout of the ilvA and ilvE genes in the branched-chain α-ketoacid (the substrate of the branched-chain α-ketoacid dehydrogenase complex) synthesis pathway, the yield of 3-hydroxypropionic acid could be further improved, and the yield of 3-hydroxypropionic acid could be greatly increased after the knockout of the ilvE gene; based on the introduction of bkdF, bkdG, bkdH and lpdA1 genes, the introduction of ppc gene could further improve the yield of 3-hydroxypropionic acid. The changing trend of the yield of 3-hydroxypropionic acid was the same as the changing trend of the synthetic amount of malonyl-CoA of the corresponding strains in Examples 1 and 2.

TABLE 3
List of primers

| Primer | Sequence | Used in |
|---|---|---|
| mcr-F | 5′-GCTAACAGGAGGAATTAACCATGG | Step |
| | GCAGCAGCCATCACCATCATC-3′ | (5) |
| | (SEQ ID NO: 55) | |
| mcr-R | 5′-ACTAGTACCAGATCTACCCTTTAC | Step |
| | ACGGTAATCGCCCGTCCGCGA-3′ | (4) |
| | (SEQ ID NO: 56) | |
| F-105 | 5′-TAGCATTTTTATCCATAAGATT | Step |
| | AGC-3 ′ (SEQ ID NO: 57) | (4) |

Example 4. Expression of Streptomyces avermitilis branched-chain α-ketoacid dehydrogenase complex gene bkdFGH-lpdA1 can increase the yield of Humulus lupulus β acid precursor PIVP

Heterologous expression of type II polyketide picric acid derived from the plant Humulus lupulus in E. coli

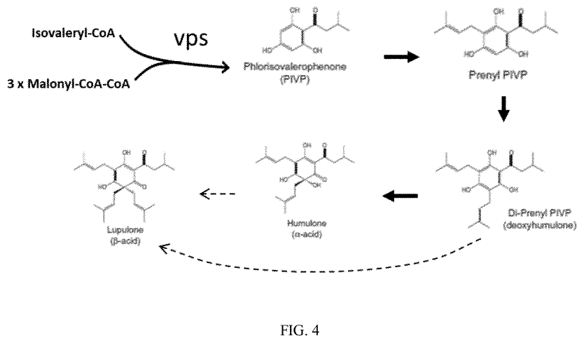

Picric acid, as a flavor substance of Humulus lupulus (belonging to the genus Humulus, family Cannabaceae), is specifically synthesized and accumulated in the glandular hairs of Humulus lupulus. It is an essential element in the beer brewing industry. It has high medicinal value and health care functions, and is also a precursor substance of many drugs. It has now been reported that its pathway synthesis can be achieved in yeast. The main pathway is branched-chain fatty acyl-CoA and malonyl-CoA under the action of vps (valerophenone synthase) to generate 3-methyl-isobutyrylphloroglucinol (PIVP), and then PIVP and DMAPP under the action of HIPT1 HIPT2 (prenyltransferase) to generate direct precursor Di-Prenyl PIVP. Di-Prenyl PIVP then undergoes an oxidation reaction to generate picric acid (FIG. 4). In this example, Humulus lupulus valerophenone synthase gene, vps gene, was introduced into M-ABC, M-FGH, M-opFGH, MA-FGH, ME-FGH, and M-FGH-ppc obtained in Example 1 and Example 2 to synthesize PIVP, and the BW of Example 1 was used as a control. The primers used are shown in Table 4 and Table 3.

(8) Construction of a plasmid expressing Humulus lupulus valerophenone synthase gene vps gene

(8-a) The nucleotide sequence of the Humulus lupulus valerophenone synthase gene vps gene is shown in SEQ ID NO: 25 in the sequence listing. The vps gene was synthesized by the whole gene synthesis and ligated to the vector pUC57 to obtain the vector pUC57-vps. The vps gene fragment was amplified by PCR using pUC57-vps as the template and vps-F/vps-R as primers.

(8-b) The PCR amplified fragment obtained in above step (8-a) was subjected to agarose gel electrophoresis to recover the target fragment; at the same time, the vector pLB1a (the nucleotide sequence of the vector pLB1a is shown in SEQ ID NO: 24) was digested with NcoI and XhoI, and the large vector fragment LB1a-NX (i.e., the vector backbone) was recovered. The above recovered target fragment was ligated with the LB1a-NX fragment by the Gibson assembly method, and the ligated product was transformed into Escherichia coli DH5α competent cells (TransGen Biotech, item number: CD201) by the CaCl₂ method. The cells were then spread on a LB plate containing streptomycin, and cultured at 37° C. overnight. Clones were picked and the clones from which the target fragment could be amplified with primers F-105/vps-R were identified and sequenced. A positive clone was screened and the plasmid was extracted, and the obtained positive plasmid with the correct sequence was named pLB1a-vps.

pLB1a-vps contains the vps gene shown in SEQ ID NO: 25 in the sequence listing, and can express the vps protein shown in SEQ ID NO: 26.

(9) Construction of PIVP-producing strain and whole-cell catalysis of 3-hydroxypropionic acid

(9-a) The vector pLB1a-vps obtained in step (8) was introduced into M-ABC, M-FGH, M-opFGH, MA-FGH, ME-FGH, M-FGH-ppc and BW respectively, to obtain recombinant strains, which were named M-ABC-PIVP, M-FGH-PIVP, M-opFGH-PIVP, MA-FGH-PIVP, ME-FGH-PIVP, M-FGH-ppc-PIVP and BW-PIVP. Each recombinant strain was used as the test strain for the production of PIVP.

(9-b) Cultivation of Engineered Strains and Induction of Enzymes

Each overnight cultured test strain was inoculated into 20 ml of medium B described in step (3-b) in a shake flask at an inoculum concentration of 1%; after culturing at 37° C. for 6 h, arabinose was added to the culture system to a final mass percentage of 0.2%; the cultivation was continued for 12 h, and the cells were collected.

(9-c) Whole-Cell Catalysis of PIVP

The cells collected above were resuspended in 5 ml of medium C in a shake flask; after culturing at 37° C. for 8 h, the cells were collected by centrifugation, and then were resuspended in 400 μL of 80% (volume percent) methanol aqueous solution pre-cooled at −80° C.; the cells were disrupted by sonication, centrifuged at 12,000 rpm at 4° C. for 20 min, and the supernatant was collected to detect the content of PIVP in the supernatant. The amount of the cells used was 5 mL of cell suspension with an OD600 of 30. The content of PIVP in the supernatant was analyzed by LCMS/MS using the standard curve method (external standard method) with PIVP (TRC, P339590-1 g) as the standard.

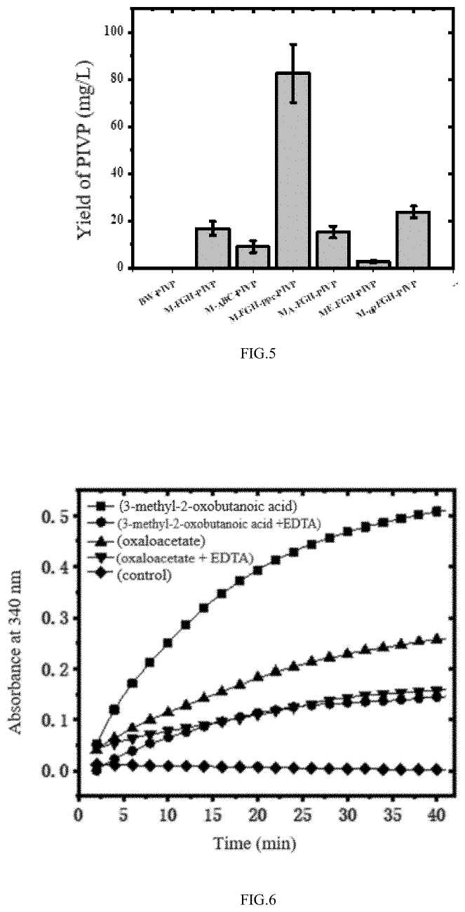

The results are shown in FIG. 5. The contents of PIVP in the supernatants obtained from M-ABC-PIVP, M-FGH-PIVP, M-opFGH-PIVP. MA-FGH-PIVP, ME-FGH-PIVP, M-FGH-ppc-PIVP and BW-PIVP were 8.96, 16.68, 23.63, 15.14, 2.49, 82.50 and 0 mg/L, respectively. BW-PIVP did not produce PIVP, M-ABC-PIVP, M-FGH-PIVP, M-opFGH-PIVP, MA-FGH-PIVP, ME-FGH-PIVP, M-FGH-ppc-PIVP can all produce PIVP.

The results showed that PIVP could be produced after the gene encoding branched-chain α-ketoacid dehydrogenase complex was introduced into E. coli: BW-PIVP did not synthesize PIVP; compared with the introduction of bkdA, bkdB, bkdC and lpdA1 genes, the introduction of bkdF, bkdG, bkdH and lpdA1 genes results in a higher yield of PIVP; while bkdH and lpdA1 genes were optimized according to the codon preference of E. coli, the yield of PIVP could be further improved; based on the introduction of bkdF, bkdG, bkdH and lpdA1 genes, the introduction of ppc gene could greatly improve the yield of PIVP. Based on the introduction of bkdF, bkdG, bkdH and lpdA1 genes, knockout of the ilvA and ilvE genes in the branched-chain α-ketoacid (the substrate of the branched-chain α-ketoacid dehydrogenase complex) synthesis pathway, the yield of PIVP did not further increase as the changing trend of the synthetic amount of malonyl-CoA of the corresponding strain in Example 1. This is because branched-chain α-ketoacids are required in the production process of PIVP, while after the knockout of ilvA and ilvE genes, the content of branched-chain α-ketoacids decreased, which in turn affected the yield of PIVP. Therefore, in the production of target products with malonyl-CoA as an intermediate product, it can be determined whether to knock out genes in the branched-chain α-ketoacid synthesis pathway according to whether branched-chain α-ketoacids are required in the synthetic pathway.

TABLE 4
List of primers

| | | Used |
|---|---|---|
| Primer | Sequence | in |
| vps-F | 5′-GCTAACAGGAGGAATTAACCAT | Step |
| | GGCGTCCGTAACTGTAGAGC-3′ | (8) |
| | (SEQ ID NO: 58) | |
| vps-R | 5′-TAGTACCAGATCTACCCTCGA | Step |
| | GTTAGACGTTTGTGGGCACGCTGTG | (8) |
| | CA-3′ | |
| | (SEQ ID NO: 59) | |

Example 5. Expression and purification of Streptomyces avermitilis branched-chain α-ketoacid dehydrogenase complex gene and detection of its oxaloacetate dehydrogenase complex activity

(10) Construction of Streptomyces avermitilis branched-chain α-ketoacid dehydrogenase complex protein expression vector

(10-a) Using the plasmid pYB1k-bkdFGH-lpdA1 described in step (1-b) as the template, YK-BCDH-His DNA fragment was obtained by PCR amplification with primers BCDH-His-F and BCDH-His-R (Table 5).

(10-b) The YK-BCDH-His DNA fragment obtained by PCR amplification in above step (10-a) was digested with DpnI, and the digested product was transformed into Escherichia coli DH5α competent cells (TransGen Biotech, item number: CD201) by the CaCl₂ method, The cells were then spread on a LB plate containing kanamycin, and cultured at 37° C. overnight. Clones were picked and the clones from which the target fragment could be amplified with primers F-108/lpdA1-XhoI were identified and sequenced. A positive clone was screened and the plasmid was extracted, and the obtained positive plasmid with the correct sequence was named pYB1k-His-BCDH.

(11) Protein expression and purification of branched-chain α-ketoacid dehydrogenase complex from Streptomyces avermitilis

(11-a) pYB1k-His-BCDH was introduced into Escherichia coli BW25113 of Example 1, and the obtained recombinant strain was named His-BCDH.

(11-b) The overnight cultured engineered strain His-BCDH was inoculated into 5 L of medium B described in step (3-a) in a shake flask at an inoculum concentration of 1%; after culturing at 30° C. for 6 h, arabinose was added to the culture system to a final mass percentage of 0.2%; the cultivation was continued for 20 h, and the cells were collected; the cells were washed with buffer D twice, then resuspended in buffer D; the cells were disrupted, and centrifuged at 20,000 rpm for 2 h and the supernatant was collected.

Buffer D is composed of a solvent and solutes, the solvent is water, and the solutes and their concentrations in buffer D are 50 mM Tris-HCl and 200 mM KCl, pH=8.0.

(11-c) A nickel column was equilibrated with 10 column volumes of buffer D, and the supernatant obtained by centrifugation in step (11-b) was loaded onto the equilibrated nickel column; the nickel column was washed by 10 column volumes of buffer D, followed by 5 column volumes of mixed buffers with a volume ratio of buffer D to buffer E of 49/1, 45/5 and 4218, and then eluted with a mixed buffer with a volume ratio of D buffer to E buffer of 1/1, to obtain Streptomyces avermitilis branched-chain α-ketoacid dehydrogenase complex protein. The above complex protein was washed and concentrated using a 100 kDa ultrafiltration tube (Amicon Ultra-15) to obtain a desalted protein, which was used for in vitro enzyme activity assay.

Buffer E is a solution with imidazole concentration of 500 mM obtained by adding imidazole to buffer D.

(12) Detection of the activity of Streptomyces avermitilis branched-chain α-ketoacid dehydrogenase complex to catalyze the production of malonyl-CoA from oxaloacetate

In a 96-well plate, 200 μL of buffer F was added to each well, and then the wells were divided into five groups, i.e., OAA group, OAA-EDTA group, KIV group (positive control), KIV-EDTA group and control group, each with 3 replicates.

Buffer F is composed of a solvent and solutes; the solvent is 50 mM Tris-HCl buffer (pH=7.0), and the solutes and their concentrations in buffer F are 0.1 mM CoA (coenzyme A), 0.2 mM DTT (dithiothreitol), 0.2 mM TPP (thiamine pyrophosphate), 1 mM MgSO₄ and 2 mM NAD⁺ (oxidized nicotinamide adenine dinucleotide).

To each well of the OAA group, oxaloacetate was added, and the concentration of oxaloacetate in the reaction system was 3 mM;

to each well of the OAA-EDTA group, oxaloacetate and disodium ethylenediaminetetraacetate (EDTA) were added and the concentrations of oxaloacetate and EDTA in the reaction system were 3 mM and 10 mM, respectively;

to each well of the KIV group, ca-ketoisovaleric acid (3-methyl-2-oxobutanoic acid) was added and the concentration of a-ketoisovaleric acid in the reaction system was 3 mM;

to each well of the KIV-EDTA group, α-ketoisovaleric acid and EDTA were added, and the concentrations of α-ketoisovaleric acid and EDTA in the reaction system were 3 mM and 10 mM, respectively.

The control group contained buffer F only.

After the addition of each reagent in each group, the branched-chain α-ketoacid dehydrogenase complex was added, and 10 μL of 0.054 mg/mL branched-chain α-ketoacid dehydrogenase complex solution was added to each 200 μL reaction system.

Then, each group of the 96-well plate was placed at 30° C. for 30 min, and the absorbance at 340 nm was detected once per minute with a microplate reader (BioTek).

The results are shown in FIG. 6. In vitro biochemical experiments confirmed that the branched-chain α-ketoacid dehydrogenase complex derived from Streptomyces avermitilis has the activity of catalyzing the formation of malonyl-CoA from oxaloacetate. The enzymatic activity of the branched-chain α-ketoacid dehydrogenase complex was 2.238 mM/min/mg protein, and the enzymatic activity of the branched-chain α-ketoacid dehydrogenase complex was defined as the moles of NADH generated by per milligram of the branched-chain α-ketoacid dehydrogenase complex per minute.

TABLE 5
List of primers

| | | Used |
|---|---|---|
| Primer | Sequence | in |
| His-BCDH-F | 5′-CTGACATGCATCATCATCATCAT | Step |
| | CACGCCGAGAAGATGGCGATCGC-3′ | (10) |
| | (SEQ ID NO: 60) | |
| His-BCDH-R | 5′-CATGTCAGTTACCCCCCTGTCC-3′ | Step |
| | (SEQ ID NO: 61) | (10) |

### INDUSTRIAL APPLICATION

The present invention discovers a new source of malonyl-CoA, that is, malonyl-CoA can be obtained by catalyzing oxaloacetate by a branched-chain α-ketoacid dehydrogenase complex. Through biochemical and genetic tests, the branched-chain α-ketoacid dehydrogenase complex was proved to have oxaloacetate dehydrogenase activity. In addition, the present invention also finds that introducing/improving phosphoenolpyruvate carboxylase can further increase the synthetic amount of malonyl-CoA, and knocking out genes in the branched-chain α-ketoacid synthesis pathway can also increase synthetic amount of malonyl-CoA. The present invention further utilizes the branched-chain α-ketoacid dehydrogenase complex to prepare the target products with malonyl-CoA as an intermediate product, such as 3-hydroxypropionic acid, picric acid or an intermediate from malonyl-CoA to picric acid in the picric acid synthesis pathway. It is shown that the branched-chain α-ketoacid dehydrogenase complex of the present invention can be used to prepare malonyl-CoA and target products with malonyl-CoA as an intermediate product, such as 3-hydroxypropionic acid, picric acid, fatty acids, polyketides and flavonoids, etc., and thus has broad application prospects.

1-22. (canceled)

23. A method for preparing malonyl-CoA, comprising the following steps 11) and 12):
11) introducing a gene encoding a branched-chain α-ketoacid dehydrogenase complex into a biological cell strain to obtain a recombinant cell strain capable of expressing the gene encoding the branched-chain α-ketoacid dehydrogenase complex, which was named recombinant cell strain A;
12) culturing the recombinant cell strain A to prepare malonyl-CoA.

24. The method according to claim 23, wherein the branched-chain α-ketoacid dehydrogenase complex is the following M1) or M2):
M1) a set of proteins consisting of a bkdF protein, a bkdG protein, a bkdH protein and a lpdA1 protein;
M2) a set of proteins consisting of a bkdA protein, a bkdB protein, a bkdC protein and the lpdA1 protein;
the gene encoding the branched-chain α-ketoacid dehydrogenase complex is the following L1) or L2):
L1) a set of genes consisting of a gene encoding the bkdF protein, a gene encoding the bkdG protein, a gene encoding the bkdH protein and a gene encoding the lpdA1 protein;
L2) a set of genes consisting of a gene encoding the bkdA protein, a gene encoding the bkdB protein, a gene encoding the bkdC protein and a gene encoding the lpdA1 protein.

25. The method according to claim 24, wherein the bkdF protein, the bkdG protein, the bkdH protein, the lpdA1 protein, the bkdA protein, the bkdB protein and the bkdC protein and their encoding genes are derived from Streptomyces avermitilis.

26. The method according to claim 24, wherein the bkdF protein is the following a1) or a2) protein:
a1) a protein having the amino acid sequence shown in SEQ ID NO: 10 in the sequence listing;
a2) a protein having 75% or more identity with and the same function as the amino acid sequence shown in SEQ ID NO: 10, which is obtained by performing substitution and/or deletion and/or addition of one or more amino acid residues on the amino acid sequence shown in SEQ ID NO: 10 in the sequence listing;
the bkdG protein is the following a3) or a4) protein:
a3) a protein having the amino acid sequence shown in SEQ ID NO: 11 in the sequence listing;
a4) a protein having 75% or more identity with and the same function as the amino acid sequence shown in SEQ ID NO: 11, which is obtained by performing substitution and/or deletion and/or addition of one or more amino acid residues on the amino acid sequence shown in SEQ ID NO: 11 in the sequence listing;
the bkdH protein is the following a5) or a6) protein:
a5) a protein having the amino acid sequence shown in SEQ ID NO: 12 in the sequence listing;
a6) a protein having 75% or more identity with and the same function as the amino acid sequence shown in SEQ ID NO: 12, which is obtained by performing substitution and/or deletion and/or addition of one or more amino acid residues on the amino acid sequence shown in SEQ ID NO: 12 in the sequence listing;
the lpdA1 protein is the following a7) or a8) protein:
a7) a protein having the amino acid sequence shown in SEQ ID NO: 13 in the sequence listing;
a8) a protein having 75% or more identity with and the same function as the amino acid sequence shown in SEQ ID NO: 13, which is obtained by performing substitution and/or deletion and/or addition of one or more amino acid residues on the amino acid sequence shown in SEQ ID NO: 13 in the sequence listing;
the bkdA protein is the following a9) or a10) protein:
a9) a protein having the amino acid sequence shown in SEQ ID NO: 7 in the sequence listing;
a10) a protein having 75% or more identity with and the same function as the amino acid sequence shown in SEQ ID NO: 7, which is obtained by performing substitution and/or deletion and/or addition of one or more amino acid residues on the amino acid sequence shown in SEQ ID NO: 7 in the sequence listing;
the bkdB protein is the following a11) or a12) protein:
a11) a protein having the amino acid sequence shown in SEQ ID NO: 8 in the sequence listing;
a12) a protein having 75% or more identity with and the same function as the amino acid sequence shown in SEQ ID NO: 8, which is obtained by performing substitution and/or deletion and/or addition of one or more amino acid residues on the amino acid sequence shown in SEQ ID NO: 8 in the sequence listing;
the bkdC protein is the following a13) or a14) protein:
a13) a protein having the amino acid sequence shown in SEQ ID NO: 9 in the sequence listing;
a14) a protein having 75% or more identity with and the same function as the amino acid sequence shown in SEQ ID NO: 9, which is obtained by performing substitution and/or deletion and/or addition of one or more amino acid residues on the amino acid sequence shown in SEQ ID NO: 9 in the sequence listing.

27. The method according to claim 24, wherein:
the gene encoding the bkdF protein is the following b1) or b2):
b1) a DNA molecule having the nucleotide sequence shown in positions 1-1221 of SEQ ID NO: 2 in the sequence listing;
b2) a DNA molecule having 75% or more identity with and the same function as the nucleotide sequence defined in b1);
the gene encoding the bkdG protein is the following b3) or b4):
b3) a DNA molecule having the nucleotide sequence shown in positions 1223-2200 of SEQ ID NO: 2 in the sequence listing;
b4) a DNA molecule having 75% or more identity with and the same function as the nucleotide sequence defined in b3);
the gene encoding the bkdH protein is the following b5) or b6) or b7):
b5) a DNA molecule having the nucleotide sequence shown in SEQ ID NO: 3 in the sequence listing;
b6) a DNA molecule having the nucleotide sequence shown in positions 2220-3608 of SEQ ID NO: 2 in the sequence listing;
b7) a DNA molecule having 75% or more identity with and the same function as the nucleotide sequence defined in b5) or b6);
the gene encoding the lpdA1 protein is the following b8) or b9) or b10):
b8) a DNA molecule having the nucleotide sequence shown in SEQ ID NO: 5 in the sequence listing;
b9) a DNA molecule having the nucleotide sequence shown in SEQ ID NO: 4 in the sequence listing;
b10) a DNA molecule having 75% or more identity with and the same function as the nucleotide sequence defined in b8) or b9);
the gene encoding the bkdA protein is the following b11) or b12):
b11) a DNA molecule having the nucleotide sequence shown in positions 1-1146 of SEQ ID NO: 1 in the sequence listing;
b12) a DNA molecule having 75% or more identity with and the same function as the nucleotide sequence defined in b11);
the gene encoding the bkdB protein is the following b13) or b14):
b13) a DNA molecule having the nucleotide sequence shown in positions 1220-2224 of SEQ ID NO: 1 in the sequence listing;
b14) a DNA molecule having 75% or more identity with and the same function as the nucleotide sequence defined in b13);
the gene encoding the bkdC protein is the following b15) or b16):
b15) a DNA molecule having the nucleotide sequence shown in positions 2224-3591 of SEQ ID NO: 1 in the sequence listing;
b16) a DNA molecule having 75% or more identity with and the same function as the nucleotide sequence defined in b15).

28. The method according to claim 23, wherein the biological cell strain contains a branched-chain α-ketoacid synthesis pathway, and step 11) can further comprise the step of inhibiting the synthesis of branched-chain α-ketoacids in the biological cell strain.

29. The method according to claim 28, wherein the step of “inhibiting the synthesis of branched-chain α-ketoacids” is achieved by “knocking out at least one gene in the branched-chain α-ketoacid synthesis pathway in the biological cell strain, or reducing the content or the activity of at least one protein encoded by the genes in the branched-chain α-ketoacid synthesis pathway”.

30. The method according to claim 28, wherein the step of “inhibiting the synthesis of branched-chain α-ketoacids” is achieved by “knocking out the ilvA gene or/and the ilvE gene in the biological cell strain, or reducing the content or the activity of the proteins encoded by the ilvA gene or/and the ilvE gene in the biological cell strain”.

31. The method according to claim 30, wherein the ilvA gene encodes the following a15) or a16) protein:
a15) a protein having the amino acid sequence shown in SEQ ID NO: 15 in the sequence listing;
a16) a protein having 75% or more identity with and the same function as the amino acid sequence shown in SEQ ID NO: 15, which is obtained by performing substitution and/or deletion and/or addition of one or more amino acid residues on the amino acid sequence shown in SEQ ID NO: 15 in the sequence listing;
the ilvE gene encodes the following a17) or a18) protein:
a17) a protein having the amino acid sequence shown in SEQ ID NO: 17 in the sequence listing;
a18) a protein having 75% or more identity with and the same function as the amino acid sequence shown in SEQ ID NO: 17, which is obtained by performing substitution and/or deletion and/or addition of one or more amino acid residues on the amino acid sequence shown in SEQ ID NO: 17 in the sequence listing;
further,
the ilvA gene is the following b17) or b18):
b17) a DNA molecule having the nucleotide sequence shown in SEQ ID NO: 14 in the sequence listing;
b18) a DNA molecule having 75% or more identity with and the same function as the nucleotide sequence defined in b17);
the ilvE gene is the following b19) or b20):
b19) a DNA molecule having the nucleotide sequence shown in SEQ ID NO: 16 in the sequence listing;
b20) a DNA molecule having 75% or more identity with and the same function as the nucleotide sequence defined in b19).

32. The method according to claim 23, wherein step 11) further comprises the step of introducing a gene encoding a ppc protein into the biological cell strain and expressing the encoding gene, or increasing the content of the ppc protein or enhancing the activity of the ppc protein in the biological cell strain;
further, the ppc protein and its encoding gene are derived from Corynebacterium glutamicum;
still further, the ppc protein is the following a19) or a20):
a19) a protein having the amino acid sequence shown in SEQ ID NO: 19 in the sequence listing;
a20) a protein having 75% or more identity with and the same function as the amino acid sequence shown in SEQ ID NO: 19, which is obtained by performing substitution and/or deletion and/or addition of one or more amino acid residues on the amino acid sequence shown in SEQ ID NO: 19 in the sequence listing;
the gene encoding the ppc protein is the following b21) or b22):
b21) a DNA molecule having the nucleotide sequence shown in SEQ ID No: 18 in the sequence listing;
b22) a DNA molecule having 75% or more identity with and the same function as the nucleotide sequence defined in b21).

33. The method according to claim 23, wherein the biological cell strain can express outer membrane protease VII, and step 11) further comprises the step of knocking out the gene encoding the outer membrane protease VII in the biological cell strain or reducing the content or the activity of the outer membrane protease VII in the biological cell strain;
further, the outer membrane protease VII is an ompT protein;
still further, the ompT protein is the following a21) or a22):
a21) a protein having the amino acid sequence shown in SEQ ID NO: 28 in the sequence listing;
a22) a protein having 75% or more identity with and the same function as the amino acid sequence shown in SEQ ID NO: 28, which is obtained by performing substitution and/or deletion and/or addition of one or more amino acid residues on the amino acid sequence shown in SEQ ID NO: 28 in the sequence listing;
the gene encoding the ompT protein is the following b23) or b24):
b23) a DNA molecule having the nucleotide sequence shown in SEQ ID NO: 27 in the sequence listing;
b24) a DNA molecule having 75% or more identity with and the same function as the nucleotide sequence defined in b23).

34. The method according to claim 23, wherein the biological cell strain contains oxaloacetate synthesis pathway and can synthesize oxaloacetate;
further, the biological cell strain is a microbial cell strain, an animal cell strain or a plant cell strain;
still further, the microbial cell strain is N1) or N2) or N3):
N1) bacteria or fungi;
N2) Escherichia coli;
N3) Escherichia coli BW25113.

35. Any of the following methods:
( ). A method for preparing malonyl-CoA, comprising: using the branched-chain α-ketoacid dehydrogenase complex in claim 23 to carry out a catalytic reaction to obtain malonyl-CoA from the substrate oxaloacetate; or
( ). A method for producing a target product with malonyl-CoA as an intermediate product, comprising: culturing the recombinant cell strain A to prepare the target product.

36. The method according to claim 35, wherein the catalytic reaction is carried out in buffer F; the buffer F is composed of a solvent and solutes, and the solvent is 50 mM Tris-HCl buffer (pH=7.0), and the solutes and their concentrations in the buffer F are as follows: 0.1 mM coenzyme A, 0.2 mM dithiothreitol, 0.2 mM triphenyl phosphate, 1 mM MgSO₄, and 2 mM NAD⁺;
or/and, the catalytic reaction is carried out at 30-37° C.;
further, the catalytic reaction is carried out at 30° C.

37. The method according to claim 35, wherein the target product is 3-hydroxypropionic acid, and the method comprises the steps of: introducing into the recombinant cell strain A a gene encoding a mcr protein and expressing the encoding gene or increasing the content of the mcr protein or enhancing the activity of the mcr protein in the recombinant cell strain A, to obtain a recombinant cell strain, which is recorded as recombinant cell strain-mcr; culturing the recombinant cell strain-mcr to prepare the target product;
further, the mcr protein and its encoding gene are derived from Chloroflexus aurantiacus;
still further, the mcr protein is composed of an mcr N-terminal domain and an mcr C-terminal domain, and the mcr N-terminal domain is the following a23) or a24):
a23) a protein having the amino acid sequence shown in SEQ ID NO: 22 in the sequence listing;
a24) a protein having 75% or more identity with and the same function as the amino acid sequence shown in SEQ ID NO: 22, which is obtained by performing substitution and/or deletion and/or addition of one or more amino acid residues on the amino acid sequence shown in SEQ ID NO: 22 in the sequence listing;
the mcr C-terminal domain is the following a25) or a26):
a25) a protein having the amino acid sequence shown in SEQ ID NO: 23 in the sequence listing;
a26) a protein having 75% or more identity with and the same function as the amino acid sequence shown in SEQ ID NO: 23, which is obtained by performing substitution and/or deletion and/or addition of one or more amino acid residues on the amino acid sequence shown in SEQ ID NO: 23 in the sequence listing;
the gene encoding the mcr protein is composed of the gene encoding the mcr N-terminal domain and the gene encoding the mcr C-terminal domain, and the gene encoding the mcr N-terminal domain is the following b25) or b26):
b25) a DNA molecule having the nucleotide sequence shown in positions 1-1689 of SEQ ID NO: 21 in the sequence listing;
b26) a DNA molecule having 75% or more identity with and the same function as the nucleotide sequence defined in b25);
the gene encoding the mcr C-terminal domain is the following b27) or b28):
b27) a DNA molecule having the nucleotide sequence shown in positions 1704-3749 of SEQ ID NO: 21 in the sequence listing;
b28) a DNA molecule having 75% or more identity with and the same function as the nucleotide sequence defined in b27);
still further, the gene encoding the mcr protein is the following b29) or b30):
b29) a DNA molecule having the nucleotide sequence shown in SEQ ID NO: 21 in the sequence listing;
b30) a DNA molecule having 75% or more identity with and the same function as the nucleotide sequence defined in b29).

38. The method according to claim 37, wherein the target product picric acid or an intermediate product from malonyl-CoA to picric acid in the picric acid synthesis pathway, and the method comprises the steps of: introducing into the recombinant cell strain A a gene encoding a vps protein and expressing the encoding gene, or increasing the content of the vps protein or enhancing the activity of the vps protein in the recombinant cell strain A, to obtain a recombinant cell strain, which is recorded as recombinant cell strain-vps; culturing the recombinant cell strain-vps to prepare the target product.
further, the vps protein and its encoding gene are derived from Humulus lupulus;
still further, the vps protein is the following a27) or a28):
a27) a protein having the amino acid sequence shown in SEQ ID NO: 26 in the sequence listing;
a28) a protein having 75% or more identity with and the same function as the amino acid sequence shown in SEQ ID NO: 26, which is obtained by performing substitution and/or deletion and/or addition of one or more amino acid residues on the amino acid sequence shown in SEQ ID NO: 26 in the sequence listing;
the gene encoding the vps protein is the following b31) or b32):
b31) a DNA molecule having the nucleotide sequence shown in SEQ ID NO: 25 in the sequence listing;
b32) a DNA molecule having 75% or more identity with and the same function as the nucleotide sequence defined in b31).

39. A reagent set, which is reagent set A or reagent set B or reagent set C or reagent set D;
the reagent set A includes the branched-chain α-ketoacid dehydrogenase complex or the gene encoding the branched-chain α-ketoacid dehydrogenase complex in claim 1;
the reagent set B consists of the reagent set A and the mcr protein or the gene encoding the mcr protein;
the reagent set C consists of the reagent set A and the vps protein or the gene encoding the vps protein;
the reagent set D consists of a recombinant cell strain, which is the recombinant cell strain A, the recombinant cell strain-mcr or the recombinant cell strain-vps.

40. The reagent set according to claim 39, wherein the reagent set A further includes the ppc protein or the gene encoding the ppc protein.

41. The reagent set according to claim 39, wherein the reagent set A also includes a substance that inhibits the synthesis of branched-chain α-ketoacids.


SEQUENCE LISTING (61 sequences)

SEQ ID NO: 1 - DNA, 3591 bp, Artificial Sequence
  Synthesized
atgacggtcatggagcagcggggcgcttaccggcccacaccgccgcccgcctggcagccccgcaccgaccccgcgccactgctgcccgacgcgctgccccaccgcgtcctgggcaccgaggcggccgcggaggccgacccgctactgctgcgccgcctgtacgcggagctggtgcgcggccgccgctacaacacgcaggccacggctctcaccaagcagggccggctcgccgtctacccgtcgagcacgggccaggaggcctgcgaggtcgccgccgcgctcgtgctggaggagcgcgactggctcttccccagctaccgggacaccctcgccgccgtcgcccgcggcctcgatcccgtccaggcgctcaccctcctgcgcggcgactggcacaccgggtacgacccccgtgagcaccgcatcgcgcccctgtgcacccctctcgcgacccagctcccgcacgccgtcggcctcgcgcacgccgcccgcctcaagggcgacgacgtggtcgcgctcgccctggtcggcgacggcggcaccagcgagggcgacttccacgaggcactgaacttcgccgccgtctggcaggcgccggtcgtcttcctcgtgcagaacaacggcttcgccatctccgtcccgctcgccaagcagaccgccgccccgtcgctggcccacaaggccgtcggctacgggatgccgggccgcctggtcgacggcaacgacgcggcggccgtgcacgaggtcctcagcgacgccgtggcccacgcgcgcgcgggaggggggccgacgctcgtggaggcggtgacctaccgcatcgacgcccacaccaacgccgacgacgcgacgcgctaccggggggactccgaggtggaggcctggcgcgcgcacgacccgatcgcgctcctggagcacgagttgaccgaacgcgggctgctcgacgaggacggcatccgggccgcccgcgaggacgccgaggcgatggccgcggacctgcgcgcacgcatgaaccaggatccggccctggaccccatggacctgttcgcccatgtgtatgccgagcccaccccccagctgcgggagcaggaagcccagttgcgggccgagctggcagcggaggccgacgggccccaaggagtcggccgatgaagagagttgaccatcgggccccgagaagcgggccgatgacctccgttggcctttggccggaaggagccgggcgatgaccaccgttgccctcaagccggccaccatggcgcaggcactcacacgcgcgttgcgtgacgccatggccgccgaccccgccgtccacgtgatgggcgaggacgtcggcacgctcggcggggtcttccgggtcaccgacgggctcgccaaggagttcggcgaggaccgctgcacggacacgccgctcgccgaggcaggcatcctcggcacggccgtcggcatggcgatgtacgggctgcggccggtcgtcgagatgcagttcgacgcgttcgcgtacccggcgttcgagcagctcatcagccatgtcgcgcggatgcgcaaccgcacccgcggggcgatgccgctgccgatcaccatccgtgtcccctacggcggcggaatcggcggagtcgaacaccacagcgactcctccgaggcgtactacatggcgactccggggctccatgtcgtcacgcccgccacggtcgccgacgcgtacgggctgctgcgcgccgccatcgcctccgacgacccggtcgtcttcctggagcccaagcggctgtactggtcgaaggactcctggaacccggacgagccggggaccgttgaaccgataggccgcgcggtggtgcggcgctcgggccggagcgccacgctcatcacgtacgggccttccctgcccgtctgcctggaggcggccgaggcggcccgggccgagggctgggacctcgaagtcgtcgatctgcgctccctggtgcccttcgacgacgagacggtgtgcgcgtcggtgcgccggaccggacgcgccgtcgtcgtgcacgagtcgggtggttacggcggcccgggcggggagatcgccgcgcggatcaccgagcgctgcttccaccatctggaggcgccggtgctgcgcgtcgccgggttcgacatcccgtatccgccgccgatgctggagcgccatcatctgcccggtgtcgaccggatcctggacgcggtggggcggcttcagtgggaggcggggagctgatggcccaggtgctcgagttcaagctccccgacctcggggagggcctgaccgaggccgagatcgtccgctggctggtgcaggtcggcgacgtcgtggcgatcgaccagccggtcgtcgaggtggagacggccaaggcgatggtcgaggtgccgtgcccctacgggggcgtggtcaccgcccgcttcggcgaggagggcacggaactgcccgtgggctcaccgctgttgacggtggctgtcggagctccgtcctcggtgcccgcggcgtcctcgctgtccggggcgacatcggcgtcctccgcgtcctcggtgtcatcggacgacggcgagtcgtccggcaacgtcctggtcggatacggcacgtcggccgcgcccgcgcgccggcggagggtgcggccgggccaggcggcacccgtggtgacggcaactgccgccgcggccgccacgcgcgtggcggctcccgagcggagcgacggccccgtgcccgtgatctccccgctggtccgcaggctcgcccgggagaacggcctggatctgcgggcgctggcgggctccgggcccgacgggctgatcctgaggtcggacgtcgagcaggcgctgcgcgccgcgcccactcctgcccccaccccgaccatgcctccggctcccactcctgcccccacccccgccgcggcaccccgcggcacccgcatccccctccgaggggtccgcggtgccgtcgccgacaaactctcccgcagccggcgtgagatccccgacgcgacctgctgggtggacgccgacgccacggcactcatgcacgcgcgcgtggcgatgaacgcgaccggcggcccgaagatctccctcatcgcgctgctcgccaggatctgcaccgccgcactggcccgcttccccgagctcaactccaccgtcgacatggacgcccgcgaggtcgtacggctcgaccaggtgcacctgggcttcgccgcgcagaccgaacgggggctcgtcgtcccggtcgtgcgggacgcgcacgcgcgggacgccgagtcgctcagcgccgagttcgcgcggctgaccgaggccgcccggaccggcaccctcacacccggggaactgaccggcggcaccttcacgttgaacaactacggggtgttcggcgtcgacggttccacgccgatcatcaaccaccccgaggcggccatgctgggcgtcggccgcatcatccccaagccgtgggtgcacgagggcgagctggcggtgcggcaggtcgtccagctctcgctcaccttcgaccaccgggtgtgcgacggcggcacggcaggcggtttcctgcgctacgtggcggactgcgtggaacagccggcggtgctgctgcgcaccctgtag

SEQ ID NO: 2 - DNA, 3608 bp, Artificial Sequence
  Synthesized
atgaccgtggagagcactgccgcgcgaaagccgcgacgcagcgccggtacgaagagcgccgcagccaagcgcaccagccccggcgccaagaagtcaccgagcacgaccggcgccgagcacgagctgattcagctgctcacgcccgacggccggcgggtgaagaaccccgagtacgacgcgtacgtcgcggacatcacccccgaagagctgcgcggtctgtaccgggacatggtgctgagccgccgcttcgacgcagaggccacctccctgcaacgccagggcgagctgggcctgtgggcctcgatgctcgggcaggaggccgcccagatcggctcgggccgggccacccgtgacgacgactacgtcttcccgacctaccgcgagcacggcgtcgcctggtgccgcggggtcgaccccaccaacctgctcggcatgttccgcggcgtgaacaacggcggctgggatcccaacagcaacaacttccacctctacacgatcgtcatcggctcgcagacgctgcacgccaccggctacgccatgggtatcgccaaggacggcgccgactcggccgtgatcgcgtacttcggtgacggcgcctccagccagggtgacgtcgccgaatcgttcaccttctccgcggtctacaacgcccctgtcgtcttcttctgccagaacaaccagtgggcgatctccgagcccaccgagaagcagacccgcgtcccgctctaccagcgcgcgcagggctacggcttcccgggcgtccgcgtcgacggcaacgacgtactggcctgcctcgccgtcaccaagtgggccctcgagcgggcccgccggggcgaggggcccacgttggtcgaggcgttcacgtaccgcatgggcgcgcacaccacctccgacgacccgaccaagtaccgggccgacgaggagcgcgaggcgtgggaggcgaaggacccgatcctgcgtctgcgcacgtatctcgaggcctcaaaccacgcggacgagggattcttcgcggaactcgaggtggagagcgaggcgttgggaaggcgagtgcgcgaagtggtgcgtgccatgccggacccggaccacttcgccatcttcgagaacgtgtacgcggacgggcatgcgctcgtcgacgaggagcgggcgcagttcgccgcctaccaggcgtcgttcacgacggagcctgacggcggctccgccgcgggacaggggggtaactgacatggccgagaagatggcgatcgccaaggcgatcaacgagtcgctgcgcaaggccctggagtccgaccccaaggttctgatcatgggtgaggacgtcggcaagctcggtggcgtcttccgcgtcaccgacggcctgcagaaggacttcggcgaggagcgggtcatcgacaccccgctcgccgagtcgggcatcgtcggcacggcgatcggtctcgccctgcgcggctaccgcccggtggtggagatccagttcgacggcttcgtcttcccggcgtacgaccagatcgtcacgcagctcgcgaagatgcacgcgcgggcgctcggcaagatcaagctccccgttgtcgtccacatcccgtacggcggcggcatcggcgccgtcgagcaccactccgagtcccccgaggcgctcttcgcgcacgtggcgggcctcaaggtggtctccccgtccaacgcgtcggacgcgtactggatgatgcagcaggccatccagagcgacgacccggtgatcttcttcgagtcgaagcggcgctactgggacaagggcgaggtcaacgtcgaggcgatccccgacccgctgcacaaggcccgtgtggtgcgtgagggcaccgacctgacgctcgccgcgtacggcccgatggtgaaggtctgccaggaggccgcggccgccgccgaggaggagggcaagtccctggaggtcgtcgacctgcgctccatgtcgccgatcgacttcgacgccgtccaggcctccgtcgagaagacccgccgtctggtcgtggtgcacgaggcgccggtgttcctgggcacgggcgcggagatcgccgcccgcatcacggagcgctgcttctaccacctggaggcacccgtgctgagggtcggcggctaccacgccccgtatccgccggcgcgtctggaagaggagtaccttccgggccttgaccgggtgctcgatgccgtcgaccgctcgctggcgtactgaggagagggtcgtgacgacgatgactgaggcgtccgtgcgtgagttcaagatgcccgatgtgggtgagggactcaccgaggccgagatcctcaagtggtacgtccagcccggcgacaccgtcaccgacggccaggtcgtctgcgaggtcgagaccgcgaaggcggccgtggaactccccattccgtacgacggtgtcgtacgcgaactccgtttccccgaggggacgacggtggacgtgggacaggtgatcatcgcggtggacgtggccggcgacgcaccggtggcggagatccccgtgcccgcgcaggaggctccggtccaggaggagcccaagcccgagggccgcaagcccgtcctcgtgggctacggggtggccgagtcctccaccaagcgccgtccgcgcaagagcgcgccggcgagcgagcccgctgcggagggcacgtacttcgcagcgaccgttctccagggcatccagggcgagctgaacggacacggcgcggtgaagcagcgtccgctggcgaagccgccggtgcgcaagctggccaaggacctgggcgtcgacctcgcgacgatcacgccgtcgggccccgacggcgtcatcacgcgcgaggacgtgcacgcggcggtggcgccaccgccgccggcaccccagcccgtgcagacgcccgctgccccggccccggcgccggtggccgcgtacgacacggctcgtgagacccgtgtccccgtcaagggcgtccgcaaggcgacggcggcggcgatggtcggctcggcgttcacggcgccgcacgtcacggagttcgtgacggtggacgtgacgcgcacgatgaagctggtcgaggagctgaagcaggacaaggagttcaccggcctgcgggtgaacccgctgctcctcatcgccaaggcgctcctggtcgcgatcaagcggaacccggacatcaacgcgtcctgggacgaggcgaaccaggagatcgtcctcaagcactatgtgaacctgggcatcgcggcggccaccccgcgcggtctgatcgtcccgaacatcaaggacgcccacgccaagacgctgccgcaactggccgagtcactgggtgagttggtgtcgacggcccgcgagggcaagacgtccccgacggccatgcagggcggcacggtcacgatcacgaacgtcggcgtcttcggcgtcgacacgggcacgccgatcctcaaccccggcgagtccgcgatcctcgcggtcggcgcgatcaagctccagccgtgggtccacaagggcaaggtcaagccccgacaggtcaccacgctggcgctcagcttcgaccatcgcctggtcgacggcgagctgggctccaaggtgctggccgacgtggcggcgatcctggagcagccgaagcggctgatcacctgggcctag

SEQ ID NO: 3 - DNA, 1389 bp, Artificial Sequence
  Synthesized
atgactgaagcgtccgtgcgtgagttcaaaatgccggacgtgggtgaaggtctgaccgaagcggaaatcctgaaatggtatgtgcagcctggtgacacggttaccgatggccaggttgtttgcgaggtagaaactgcgaaagcggctgttgagctgccgatcccgtacgacggcgtggtgcgtgaactgcgtttcccggaaggtactactgtcgatgtcggccaggtaattatcgcagttgatgtggccggcgacgcaccggttgcggaaatcccggtgccggcgcaggaagccccggtccaggaagagccgaaaccggaaggtcgtaaacctgtgctggtaggttatggtgttgctgaaagcagcactaaacgtcgcccgcgtaagtccgcgccagcgtccgaaccggcggcagaaggcacctatttcgctgccaccgttctgcaaggtattcagggtgaactgaacggccacggtgcagtaaaacagcgcccactggcgaaaccaccagttcgcaaactggcgaaagacctgggtgtggatctggcgactattaccccgtccggcccggatggcgttattacccgtgaagacgtacacgctgctgtggcgcctccgccgccggcacctcaaccggtgcagaccccggcggcaccggctccggctccggtggccgcgtacgatacggcgcgtgagacgcgcgttccagtaaaaggtgttcgtaaggctactgccgctgctatggtgggtagcgcgttcactgcacctcacgttaccgaatttgttacggtagatgtgactcgtactatgaaactggtggaagaactgaaacaggataaagagttcactggtctgcgcgttaacccgctgctgctgattgcgaaagcactgctggtcgctatcaagcgtaacccggacatcaatgcatcctgggacgaagcaaaccaggaaatcgttctgaagcactacgtaaacctcggtatcgcggctgcaaccccgcgcggcctgatcgtgccaaatatcaaagacgctcatgccaaaaccctgccgcagctggcggaatctctgggtgaactggtttccaccgctcgcgagggtaagacctccccgactgcaatgcagggcggtacggtcaccatcaccaatgtgggtgtattcggtgttgacaccggcacgccgatcctgaacccgggtgagtccgccatcctcgctgtaggtgctatcaaactgcaaccgtgggttcacaaaggcaaagttaaaccacgtcaggttaccaccctggctctgagcttcgaccaccgtctggttgacggtgaactgggctccaaggtactggcggacgtggcggcgatcctggagcagccgaagcgtctcatcacttgggcataa

SEQ ID NO: 4 - DNA, 1389 bp, Artificial Sequence
  Synthesized
atggcgaacgacgccagcaccgttttcgacctagtgatcctcggcggtggtagcggtggttacgccgcggccctgcgcggagcgcagctgggcctggacgtcgccctgatcgagaaggacaaggtcggcggtacctgcctgcaccgtgggtgcatccccaccaaggcgctgctgcacgcgggcgagatcgccgaccaggcccgcgagagcgagcagttcggcgtcaaggccaccttcgagggcatcgacgtaccggccgtccacaagtacaaggacggggtcatctcgggcctgtacaagggtctgcaggggctgatcgcctcccgcaaggtgacgtacatcgagggtgagggccgtctgtcctccccgacctccgtcgacgtgaacggccagcgcgtccagggccgccacgtgctcctggcgaccggctccgtgccgaagtcgctgccgggcctggcgatcgacggcaaccgcatcatctcctccgaccacgcgctggtcctggaccgcgtcccggagtccgcgatcgtgctcggcggcggcgtcatcggcgtcgagttcgcctccgcgtggaagtccttcggagccgacgtgacggtgatcgagggcctcaagcacctcgtcccggtcgaggacgagaactcctccaagcttcttgagcgcgcgttccgcaagcgcggcatcaagttcaacctgggcaccttcttctcgaaggccgagtacacccagaacggtgtcaaggtcaccctcgccgacggcaaggagttcgaggccgaggtcctgctcgtcgccgtcggccgcggcccggtctcgcagggcctcggctacgaggagcagggcgtcgccatggaccgcggctacgtcctggtcgacgagtacatgcggacgaacgtcccgaccatctccgccgtcggtgacctggtcccgacgctccagctcgcgcacgtcggcttcgccgagggcatcctggtggcggagcgtctggccggtctgaagaccgtcccgatcgactacgacggcgtgccgcgggtgacgtactgccaccccgaggtcgcctccgtgggcatcaccgaggccaaggccaaggagatctacggcgcggacaaggtcgtcgctctgaagtacaacctggcgggcaacggcaagagcaagatcctcaacaccgcgggcgagatcaagctcgtccaggtgaaggacggtgccgtggtcggcgtccacatggtcggtgaccgtatgggcgagcaggtcggcgaagcccagctgatctacaactgggaggcgctgccggccgaggtcgcccagctcatccacgcccacccgacgcagaacgaagcgatgggcgaggcccacctggccctcgcgggcaagccgctgcactcgcacgactga

SEQ ID NO: 5 - DNA, 1389 bp, Artificial Sequence
  Synthesized
atggcaaacgacgcatctacggtgttcgacctggttatcctcggtggtggttccggtggctatgctgccgcgctgcgtggtgcacagctgggtctggatgttgcgctgatcgagaaagacaaagttggtggcacttgcctgcatcgtggttgcatcccgaccaaagcgctgctgcacgcgggcgaaattgctgatcaggcacgcgaatctgaacaattcggtgtgaaagcgaccttcgagggtatcgacgttccggctgtgcacaagtacaaagatggtgttattagcggtctgtacaaaggcctccaaggtctgattgcgtcccgcaaggtgacttacatcgaaggcgagggtcgtctgtccagccctacctctgttgacgttaatggtcagcgtgttcaaggccgccacgttctgctggctaccggctctgttcctaaaagcctgccaggtctggctatcgacggtaaccgtattatctcctctgatcatgctctggtcctggaccgcgttccggagtccgcgattgttctgggtggtggcgttatcggtgttgagtttgcctctgcatggaaatctttcggcgcagatgtaaccgtaatcgaaggtctgaaacacctggttccggtcgaagacgagaactcctctaaactgctggaacgtgcattccgcaaacgcggtattaaattcaacctgggtactttcttcagcaaagcagagtatacccagaatggtgttaaagtgactctggccgacggtaaggagtttgaagccgaagttctgctggtcgcagtaggtcgtggtcctgtatctcagggtctgggctacgaagaacagggtgttgctatggaccgtggctatgttctggtcgatgagtacatgcgcaccaacgtaccgaccatctccgcagtgggtgacctggtgcctaccctgcaactggctcatgtaggtttcgcggaaggtatcctggtagctgaacgtctggcgggcctgaaaacggttccaatcgattacgatggcgttccgcgcgtgacctattgccacccggaagtggcgtctgtaggcatcaccgaagccaaagcaaaagaaatttacggtgctgacaaagtagtcgctctgaaatacaacctggcgggtaacggtaaaagcaaaatcctgaacactgctggtgaaatcaaactggttcaggttaaagacggtgcagtggtcggtgtgcacatggttggcgaccgcatgggtgaacaggtgggcgaagcacagctgatctataactgggaggctctgccggctgaagttgcgcagctgatccacgcgcacccgacccaaaacgaagctatgggcgaagctcacctggctctggctggcaagccgctgcattctcacgactaa

SEQ ID NO: 6 - DNA, 3650 bp, Artificial Sequence
  Synthesized
aatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgggtacctctcatcatcatcatcatcacagcagcggcctggtgccgcgcggcagcctcgagggtagatctggtactagtggtgaattcggtgagctcggtctgcagctggtgccgcgcggcagccaccaccaccaccaccactaatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgtcgacgcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtgcggccgccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgaccgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacttgcggagacccggtcgtcagcttgtcgtcggttcagggcagggtcgttaaatagcgcatgc

SEQ ID NO: 7 - protein, 381 aa, Artificial Sequence
  Synthesized
MTVMEQRGAYRPTPPPAWQPRTDPAPLLPDALPHRVLGTEAAAEADPLLLRRLYAELVRGRRYNTQATALTKQGRLAVYPSSTGQEACEVAAALVLEERDWLFPSYRDTLAAVARGLDPVQALTLLRGDWHTGYDPREHRIAPLCTPLATQLPHAVGLAHAARLKGDDVVALALVGDGGTSEGDFHEALNFAAVWQAPVVFLVQNNGFAISVPLAKQTAAPSLAHKAVGYGMPGRLVDGNDAAAVHEVLSDAVAHARAGGGPTLVEAVTYRIDAHTNADDATRYRGDSEVEAWRAHDPIALLEHELTERGLLDEDGIRAAREDAEAMAADLRARMNQDPALDPMDLFAHVYAEPTPQLREQEAQLRAELAAEADGPQGVGR

SEQ ID NO: 8 - protein, 334 aa, Artificial Sequence
  Synthesized
MTTVALKPATMAQALTRALRDAMAADPAVHVMGEDVGTLGGVFRVTDGLAKEFGEDRCTDTPLAEAGILGTAVGMAMYGLRPVVEMQFDAFAYPAFEQLISHVARMRNRTRGAMPLPITIRVPYGGGIGGVEHHSDSSEAYYMATPGLHVVTPATVADAYGLLRAAIASDDPVVFLEPKRLYWSKDSWNPDEPGTVEPIGRAVVRRSGRSATLITYGPSLPVCLEAAEAARAEGWDLEVVDLRSLVPFDDETVCASVRRTGRAVVVHESGGYGGPGGEIAARITERCFHHLEAPVLRVAGFDIPYPPPMLERHHLPGVDRILDAVGRLQWEAGS

SEQ ID NO: 9 - protein, 455 aa, Artificial Sequence
  Synthesized
MAQVLEFKLPDLGEGLTEAEIVRWLVQVGDVVAIDQPVVEVETAKAMVEVPCPYGGVVTARFGEEGTELPVGSPLLTVAVGAPSSVPAASSLSGATSASSASSVSSDDGESSGNVLVGYGTSAAPARRRRVRPGQAAPVVTATAAAAATRVAAPERSDGPVPVISPLVRRLARENGLDLRALAGSGPDGLILRSDVEQALRAAPTPAPTPTMPPAPTPAPTPAAAPRGTRIPLRGVRGAVADKLSRSRREIPDATCWVDADATALMHARVAMNATGGPKISLIALLARICTAALARFPELNSTVDMDAREVVRLDQVHLGFAAQTERGLVVPVVRDAHARDAESLSAEFARLTEAARTGTLTPGELTGGTFTLNNYGVFGVDGSTPIINHPEAAMLGVGRIIPKPWVHEGELAVRQVVQLSLTFDHRVCDGGTAGGFLRYVADCVEQPAVLLRTL

SEQ ID NO: 10 - protein, 406 aa, Artificial Sequence
  Synthesized
MTVESTAARKPRRSAGTKSAAAKRTSPGAKKSPSTTGAEHELIQLLTPDGRRVKNPEYDAYVADITPEELRGLYRDMVLSRRFDAEATSLQRQGELGLWASMLGQEAAQIGSGRATRDDDYVFPTYREHGVAWCRGVDPTNLLGMFRGVNNGGWDPNSNNFHLYTIVIGSQTLHATGYAMGIAKDGADSAVIAYFGDGASSQGDVAESFTFSAVYNAPVVFFCQNNQWAISEPTEKQTRVPLYQRAQGYGFPGVRVDGNDVLACLAVTKWALERARRGEGPTLVEAFTYRMGAHTTSDDPTKYRADEEREAWEAKDPILRLRTYLEASNHADEGFFAELEVESEALGRRVREVVRAMPDPDHFAIFENVYADGHALVDEERAQFAAYQASFTTEPDGGSAAGQGGN

SEQ ID NO: 11 - protein, 325 aa, Artificial Sequence
  Synthesized
MAEKMAIAKAINESLRKALESDPKVLIMGEDVGKLGGVFRVTDGLQKDFGEERVIDTPLAESGIVGTAIGLALRGYRPVVEIQFDGFVFPAYDQIVTQLAKMHARALGKIKLPVVVHIPYGGGIGAVEHHSESPEALFAHVAGLKVVSPSNASDAYWMMQQAIQSDDPVIFFESKRRYWDKGEVNVEAIPDPLHKARVVREGTDLTLAAYGPMVKVCQEAAAAAEEEGKSLEVVDLRSMSPIDFDAVQASVEKTRRLVVVHEAPVFLGTGAEIAARITERCFYHLEAPVLRVGGYHAPYPPARLEEEYLPGLDRVLDAVDRSLAY

SEQ ID NO: 12 - protein, 462 aa, Artificial Sequence
  Synthesized
MTEASVREFKMPDVGEGLTEAEILKWYVQPGDTVTDGQVVCEVETAKAAVELPIPYDGVVRELRFPEGTTVDVGQVIIAVDVAGDAPVAEIPVPAQEAPVQEEPKPEGRKPVLVGYGVAESSTKRRPRKSAPASEPAAEGTYFAATVLQGIQGELNGHGAVKQRPLAKPPVRKLAKDLGVDLATITPSGPDGVITREDVHAAVAPPPPAPQPVQTPAAPAPAPVAAYDTARETRVPVKGVRKATAAAMVGSAFTAPHVTEFVTVDVTRTMKLVEELKQDKEFTGLRVNPLLLIAKALLVAIKRNPDINASWDEANQEIVLKHYVNLGIAAATPRGLIVPNIKDAHAKTLPQLAESLGELVSTAREGKTSPTAMQGGTVTITNVGVFGVDTGTPILNPGESAILAVGAIKLQPWVHKGKVKPRQVTTLALSFDHRLVDGELGSKVLADVAAILEQPKRLITWA

SEQ ID NO: 13 - protein, 462 aa, Artificial Sequence
  Synthesized
MANDASTVFDLVILGGGSGGYAAALRGAQLGLDVALIEKDKVGGTCLHRGCIPTKALLHAGEIADQARESEQFGVKATFEGIDVPAVHKYKDGVISGLYKGLQGLIASRKVTYIEGEGRLSSPTSVDVNGQRVQGRHVLLATGSVPKSLPGLAIDGNRIISSDHALVLDRVPESAIVLGGGVIGVEFASAWKSFGADVTVIEGLKHLVPVEDENSSKLLERAFRKRGIKFNLGTFFSKAEYTQNGVKVTLADGKEFEAEVLLVAVGRGPVSQGLGYEEQGVAMDRGYVLVDEYMRTNVPTISAVGDLVPTLQLAHVGFAEGILVAERLAGLKTVPIDYDGVPRVTYCHPEVASVGITEAKAKEIYGADKVVALKYNLAGNGKSKILNTAGEIKLVQVKDGAVVGVHMVGDRMGEQVGEAQLIYNWEALPAEVAQLIHAHPTQNEAMGEAHLALAGKPLHSHD

SEQ ID NO: 14 - DNA, 1545 bp, Artificial Sequence
  Synthesized
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgacgcggtgcgcggcttcggcggcgaagtgctgctccacggcgcgaactttgatgaagcgaaagccaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgctggcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactgggcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgaattcccggaatcaccgggcgcgctgctgcgcttcctcaacacgctgggtacgtactggaacatttctttgttccactatcgcagccatggcaccgactacgggcgcgtactggcggcgttcgaacttggcgaccatgaaccggatttcgaaacccggctgaatgagctgggctacgattgccacgacgaaaccaataacccggcgttcaggttctttttggcgggttag

SEQ ID NO: 15 - protein, 514 aa, Artificial Sequence [sequence omitted]

SEQ ID NO: 16 - DNA, 930 bp, Artificial Sequence
  Synthesized
atgaccacgaagaaagctgattacatttggttcaatggggagatggttcgctgggaagacgcgaaggtgcatgtgatgtcgcacgcgctgcactatggcacttcggtttttgaaggcatccgttgctacgactcgcacaaaggaccggttgtattccgccatcgtgagcatatgcagcgtctgcatgactccgccaaaatctatcgcttcccggtttcgcagagcattgatgagctgatggaagcttgtcgtgacgtgatccgcaaaaacaatctcaccagcgcctatatccgtccgctgatcttcgtcggtgatgttggcatgggagtaaacccgccagcgggatactcaaccgacgtgattatcgctgctttcccgtggggagcgtatctgggcgcagaagcgctggagcaggggatcgatgcgatggtttcctcctggaaccgcgcagcaccaaacaccatcccgacggcggcaaaagccggtggtaactacctctcttccctgctggtgggtagcgaagcgcgccgccacggttatcaggaaggtatcgcgctggatgtgaacggttatatctctgaaggcgcaggcgaaaacctgtttgaagtgaaagatggtgtgctgttcaccccaccgttcacctcctccgcgctgccgggtattacccgtgatgccatcatcaaactggcgaaagagctgggaattgaagtacgtgagcaggtgctgtcgcgcgaatccctgtacctggcggatgaagtgtttatgtccggtacggcggcagaaatcacgccagtgcgcagcgtagacggtattcaggttggcgaaggccgttgtggcccggttaccaaacgcattcagcaagccttcttcggcctcttcactggcgaaaccgaagataaatggggctggttagatcaagttaatcaataa

SEQ ID NO: 17 - protein, 309 aa, Artificial Sequence
  Synthesized
MTTKKADYIWFNGEMVRWEDAKVHVMSHALHYGTSVFEGIRCYDSHKGPVVFRHREHMQRLHDSAKIYRFPVSQSIDELMEACRDVIRKNNLTSAYIRPLIFVGDVGMGVNPPAGYSTDVIIAAFPWGAYLGAEALEQGIDAMVSSWNRAAPNTIPTAAKAGGNYLSSLLVGSEARRHGYQEGIALDVNGYISEGAGENLFEVKDGVLFTPPFTSSALPGITRDAIIKLAKELGIEVREQVLSRESLYLADEVFMSGTAAEITPVRSVDGIQVGEGRCGPVTKRIQQAFFGLFTGETEDKWGWLDQVNQ

SEQ ID NO: 18 - DNA, 2760 bp, Artificial Sequence
  Synthesized
atgactgattttttacgcgatgacatcaggttcctcggtcaaatcctcggtgaggtaattgcggaacaagaaggccaggaggtttatgaactggtcgaacaagcgcgcctgacttcttttgatatcgccaagggcaacgccgaaatggatagcctggttcaggttttcgacggcattactccagccaaggcaacaccgattgctcgcgcattttcccacttcgctctgctggctaacctggcggaagacctctacgatgaagagcttcgtgaacaggctctcgatgcaggcgacacccctccggacagcactcttgatgccacctggctgaaactcaatgagggcaatgttggcgcagaagctgtggccgatgtgctgcgcaatgctgaggtggcgccggttctgactgcgcacccaactgagactcgccgccgcactgtttttgatgcgcaaaagtggatcaccacccacatgcgtgaacgccacgctttgcagtctgcggagcctaccgctcgtacgcaaagcaagttggatgagatcgagaagaacatccgccgtcgcatcaccattttgtggcagaccgcgttgattcgtgtggcccgcccacgtatcgaggacgagatcgaagtagggctgcgctactacaagctgagccttttggaagagattccacgtatcaaccgtgatgtggctgttgagcttcgtgagcgtttcggcgagggtgttcctttgaagcccgtggtcaagccaggttcctggattggtggagaccacgacggtaacccttatgtcaccgcggaaacagttgagtattccactcaccgcgctgcggaaaccgtgctcaagtactatgcacgccagctgcattccctcgagcatgagctcagcctgtcggaccgcatgaataaggtcaccccgcagctgcttgcgctggcagatgcagggcacaacgacgtgccaagccgcgtggatgagccttatcgacgcgccgtccatggcgttcgcggacgtatcctcgcgacgacggccgagctgatcggcgaggacgccgttgagggcgtgtggttcaaggtctttactccatacgcatctccggaagaattcttaaacgatgcgttgaccattgatcattctctgcgtgaatccaaggacgttctcattgccgatgatcgtttgtctgtgctgatttctgccatcgagagctttggattcaacctttacgcactggatctgcgccaaaactccgaaagctacgaggacgtcctcaccgagcttttcgaacgcgcccaagtcaccgcaaactaccgcgagctgtctgaagcagagaagcttgaggtgctgctgaaggaactgcgcagccctcgtccgctgatcccgcacggttcagatgaatacagcgaggtcaccgaccgcgagctcggcatcttccgcaccgcgtcggaggctgttaagaaattcgggccacggatggtgcctcactgcatcatctccatggcatcatcggtcaccgatgtgctcgagccgatggtgttgctcaaggaattcggactcatcgcagccaacggcgacaacccacgcggcaccgtcgatgtcatcccactgttcgaaaccatcgaagatctccaggccggcgccggaatcctcgacgaactgtggaaaattgatctctaccgcaactacctcctgcagcgcgacaacgtccaggaagtcatgctcggttactccgattccaacaaggatggcggatatttctccgcaaactgggcgctttacgacgcggaactgcagctcgtcgaactatgccgatcagccggggtcaagcttcgcctgttccacggccgtggtggcaccgtcggccgcggtggcggaccttcctacgacgcgattcttgcccagcccaggggggctgtccaaggttccgtgcgcatcaccgagcagggcgagatcatctccgctaagtacggcaaccccgaaaccgcgcgccgaaacctcgaagccctggtctcagccacgcttgaggcatcgcttctcgacgtctccgaactcaccgatcaccaacgcgcgtacgacatcatgagtgagatctctgagctcagcttgaagaagtacgcctccttggtgcacgaggatcaaggcttcatcgattacttcacccagtccacgccgctgcaggagattggatccctcaacatcggatccaggccttcctcacgcaagcagacctcctcggtggaagatttgcgagccatcccatgggtgctcagctggtcacagtctcgtgtcatgctgccaggctggtttggtgtcggaaccgcattagagcagtggattggcgaaggggagcaggccacccaacgcattgccgagctgcaaacactcaatgagtcctggccatttttcacctcagtgttggataacatggctcaggtgatgtccaaggcagagctgcgtttggcaaagctctacgcagacctgatcccagatacggaagtagccgagcgagtctattccgtcatccgcgaggagtacttcctgaccaagaagatgttctgcgtaatcaccggctctgatgatctgcttgatgacaacccacttctcgcacgctctgtccagcgccgatacccctacctgcttccactcaacgtgatccaggtagagatgatgcgacgctaccgaaaaggcgaccaaagcgagcaagtgtcccgcaacattcagctgaccatgaacggtctttccactgcgctgcgcaactccggctag

SEQ ID NO: 19 - protein, 919 aa, Artificial Sequence
  Synthesized
MTDFLRDDIRFLGQILGEVIAEQEGQEVYELVEQARLTSFDIAKGNAEMDSLVQVFDGITPAKATPIARAFSHFALLANLAEDLYDEELREQALDAGDTPPDSTLDATWLKLNEGNVGAEAVADVLRNAEVAPVLTAHPTETRRRTVFDAQKWITTHMRERHALQSAEPTARTQSKLDEIEKNIRRRITILWQTALIRVARPRIEDEIEVGLRYYKLSLLEEIPRINRDVAVELRERFGEGVPLKPVVKPGSWIGGDHDGNPYVTAETVEYSTHRAAETVLKYYARQLHSLEHELSLSDRMNKVTPQLLALADAGHNDVPSRVDEPYRRAVHGVRGRILATTAELIGEDAVEGVWFKVFTPYASPEEFLNDALTIDHSLRESKDVLIADDRLSVLISAIESFGFNLYALDLRQNSESYEDVLTELFERAQVTANYRELSEAEKLEVLLKELRSPRPLIPHGSDEYSEVTDRELGIFRTASEAVKKFGPRMVPHCIISMASSVTDVLEPMVLLKEFGLIAANGDNPRGTVDVIPLFETIEDLQAGAGILDELWKIDLYRNYLLQRDNVQEVMLGYSDSNKDGGYFSANWALYDAELQLVELCRSAGVKLRLFHGRGGTVGRGGGPSYDAILAQPRGAVQGSVRITEQGEIISAKYGNPETARRNLEALVSATLEASLLDVSELTDHQRAYDIMSEISELSLKKYASLVHEDQGFIDYFTQSTPLQEIGSLNIGSRPSSRKQTSSVEDLRAIPWVLSWSQSRVMLPGWFGVGTALEQWIGEGEQATQRIAELQTLNESWPFFTSVLDNMAQVMSKAELRLAKLYADLIPDTEVAERVYSVIREEYFLTKKMFCVITGSDDLLDDNPLLARSVQRRYPYLLPLNVIQVEMMRRYRKGDQSEQVSRNIQLTMNGLSTALRNSG

SEQ ID NO: 20 - DNA, 1257 bp, Artificial Sequence
  Synthesized
gtctcgagaatatcctccttataacttcgtataatgtatgctatacgaacggtaagagcgcttggccgctcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgagggcatgctgcggcagcgtgaggggatctttaccgttcgtataatgtatgctataccaagttatgaagctagcttatcaaaaagttgacaattaatcatcggctcgtataatgtgtggaaggaggaattaacc

SEQ ID NO: 21 - DNA, 3749 bp, Artificial Sequence
  Synthesized
atgggcagcagccatcaccatcatcaccacagccaggatccgagcggaacaggacgactggcaggaaagattgcgttaattaccggtggcgccggcaatatcggcagtgaattgacacgtcgctttctcgcagagggagcgacggtcattattagtggacggaatcgggcgaagttgaccgcactggccgaacggatgcaggcagaggcaggagtgccggcaaagcgcatcgatctcgaagtcatggatgggagtgatccggtcgcggtacgtgccggtatcgaagcgattgtggcccgtcacggccagatcgacattctggtcaacaatgcaggaagtgccggtgcccagcgtcgtctggccgagattccactcactgaagctgaattaggccctggcgccgaagagacgcttcatgccagcatcgccaatttacttggtatgggatggcatctgatgcgtattgcggcacctcatatgccggtaggaagtgcggtcatcaatgtctcgaccatcttttcacgggctgagtactacgggcggattccgtatgtcacccctaaagctgctcttaatgctctatctcaacttgctgcgcgtgagttaggtgcacgtggcatccgcgttaatacgatctttcccggcccgattgaaagtgatcgcatccgtacagtgttccagcgtatggatcagctcaaggggcggcccgaaggcgacacagcgcaccattttttgaacaccatgcgattgtgtcgtgccaacgaccagggcgcgcttgaacgtcggttcccctccgtcggtgatgtggcagacgccgctgtctttctggccagtgccgaatccgccgctctctccggtgagacgattgaggttacgcacggaatggagttgccggcctgcagtgagaccagcctgctggcccgtactgatctgcgcacgattgatgccagtggccgcacgacgctcatctgcgccggcgaccagattgaagaggtgatggcgctcaccggtatgttgcgtacctgtgggagtgaagtgatcatcggcttccgttcggctgcggcgctggcccagttcgagcaggcagtcaatgagagtcggcggctggccggcgcagactttacgcctcccattgccttgccactcgatccacgcgatccggcaacaattgacgctgtcttcgattgggccggcgagaataccggcgggattcatgcagcggtgattctgcctgctaccagtcacgaaccggcaccgtgcgtgattgaggttgatgatgagcgggtgctgaattttctggccgatgaaatcaccgggacaattgtgattgccagtcgcctggcccgttactggcagtcgcaacggcttacccccggcgcacgtgcgcgtgggccgcgtgtcatttttctctcgaacggtgccgatcaaaatgggaatgtttacggacgcattcaaagtgccgctatcggtcagctcattcgtgtgtggcgtcacgaggctgaacttgactatcagcgtgccagcgccgccggtgatcatgtgctgccgccggtatgggccaatcagattgtgcgcttcgctaaccgcagccttgaagggttagaatttgcctgtgcctggacagctcaattgctccatagtcaacgccatatcaatgagattaccctcaacatccctgccaacatttaacaggaggaattaacatggcagatctccatcaccatcatcaccatcacagcgccaccaccggcgcacgcagtgcatcggtcggatgggcggaaagcctgatcgggttgcatttggggaaagttgccttgattaccggtggcagcgccggtattggtgggcagatcgggcgcctcctggctttgagtggcgcgcgcgtgatgctggcagcccgtgatcggcataagctcgaacagatgcaggcgatgatccaatctgagctggctgaggtggggtataccgatgtcgaagatcgcgtccacattgcaccgggctgcgatgtgagtagcgaagcgcagcttgcggatcttgttgaacgtaccctgtcagcttttggcaccgtcgattatctgatcaacaacgccgggatcgccggtgtcgaagagatggttatcgatatgccagttgagggatggcgccataccctcttcgccaatctgatcagcaactactcgttgatgcgcaaactggcgccgttgatgaaaaaacagggtagcggttacatccttaacgtctcatcatactttggcggtgaaaaagatgcggccattccctaccccaaccgtgccgattacgccgtctcgaaggctggtcagcgggcaatggccgaagtctttgcgcgcttccttggcccggagatacagatcaatgccattgcgccgggtccggtcgaaggtgatcgcttgcgcggtaccggtgaacgtcccggcctctttgcccgtcgggcgcggctgattttggagaacaagcggctgaatgagcttcacgctgctcttatcgcggctgcgcgcaccgatgagcgatctatgcacgaactggttgaactgctcttacccaatgatgtggccgcactagagcagaatcccgcagcacctaccgcgttgcgtgaactggcacgacgttttcgcagcgaaggcgatccggcggcatcatcaagcagtgcgctgctgaaccgttcaattgccgctaaattgctggctcgtttgcataatggtggctatgtgttgcctgccgacatctttgcaaacctgccaaacccgcccgatcccttcttcacccgagcccagattgatcgcgaggctcgcaaggttcgtgacggcatcatggggatgctctacctgcaacggatgccgactgagtttgatgtcgcaatggccaccgtctattaccttgccgaccgcgtggtcagtggtgagacattccacccatcaggtggtttgcgttacgaacgcacccctaccggtggcgaactcttcggcttgccctcaccggaacggctggcggagctggtcggaagcacggtctatctgataggtgaacatctgactgaacaccttaacctgcttgcccgtgcgtacctcgaacgttacggggcacgtcaggtagtgatgattgttgagacagaaaccggggcagagacaatgcgtcgcttgctccacgatcacgtcgaggctggtcggctgatgactattgtggccggtgatcagatcgaagccgctatcgaccaggctatcactcgctacggtcgcccagggccggtcgtctgtacccccttccggccactgccgacggtaccactggtcgggcgtaaagacagtgactggagcacagtgttgagtgaggctgaatttgccgagttgtgcgaacaccagctcacccaccatttccgggtagcgcgctggattgccctgagtgatggtgcccgtctcgcgctggtcactcccgaaactacggctacctcaactaccgagcaatttgctctggctaacttcatcaaaacgacccttcacgcttttacggctacgattggtgtcgagagcgaaagaactgctcagcgcattctgatcaatcaagtcgatctgacccggcgtgcgcgtgccgaagagccgcgtgatccgcacgagcgtcaacaagaactggaacgttttatcgaggcagtcttgctggtcactgcaccactcccgcctgaagccgatacccgttacgccgggcggattcatcgcggacgggcgattaccgtgtaa

SEQ ID NO: 22 - protein, 562 aa, Artificial Sequence
  Synthesized
MGSSHHHHHHSQDPSGTGRLAGKIALITGGAGNIGSELTRRFLAEGATVIISGRNRAKLTALAERMQAEAGVPAKRIDLEVMDGSDPVAVRAGIEAIVARHGQIDILVNNAGSAGAQRRLAEIPLTEAELGPGAEETLHASIANLLGMGWHLMRIAAPHMPVGSAVINVSTIFSRAEYYGRIPYVTPKAALNALSQLAARELGARGIRVNTIFPGPIESDRIRTVFQRMDQLKGRPEGDTAHHFLNTMRLCRANDQGALERRFPSVGDVADAAVFLASAESAALSGETIEVTHGMELPACSETSLLARTDLRTIDASGRTTLICAGDQIEEVMALTGMLRTCGSEVIIGFRSAAALAQFEQAVNESRRLAGADFTPPIALPLDPRDPATIDAVFDWAGENTGGIHAAVILPATSHEPAPCVIEVDDERVLNFLADEITGTIVIASRLARYWQSQRLTPGARARGPRVIFLSNGADQNGNVYGRIQSAAIGQLIRVWRHEAELDYQRASAAGDHVLPPVWANQIVRFANRSLEGLEFACAWTAQLLHSQRHINEITLNIPANI

SEQ ID NO: 23 - protein, 681 aa, Artificial Sequence
  Synthesized
MADLHHHHHHHSATTGARSASVGWAESLIGLHLGKVALITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLILENKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRVVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARWIALSDGARLALVTPETTATSTTEQFALANFIKTTLHAFTATIGVESERTAQRILINQVDLTRRARAEEPRDPHERQQELERFIEAVLLVTAPLPPEADTRYAGRIHRGRAITV

SEQ ID NO: 24 - DNA, 4293 bp, Artificial Sequence
  Synthesized
aatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgggtacctctcatcatcatcatcatcacagcagcggcctggtgccgcgcggcagcctcgagggtagatctggtactagtggtgaattcggtgagctcggtctgcagctggtgccgcgcggcagccaccaccaccaccaccactaatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgtcgacctaattcccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgcggatcttgatgagtggatagtacgttgctaaaacatgagataaaaattgactctcatgttattggcgttaagatatacagaatgatgaggtttttttatgagactcaaggtcatgatggacgtgaacaaaaaaacgaaaattcgccaccgaaacgagctaaatcacaccctggctcaacttcctttgcccgcaaagcgagtgatgtatatggcgcttgctcccattgatagcaaggaacctcttgaacgagggcgagttttcaaaattagggctgaagaccttgcagcgctcgccaaaatcaccccatcgcttgcttatcgacaattaaaagagggtggtaagttacttggtgccagcaaaatttcgctaagaggggatgatatcattgcttcagctaaagagcttaacctgctctttactgctaaagactcccctgaagagttagatcttaacattattgagtggatagcttattcaaatgatgaaggatacttgtctttaaaattcaccagaaccatagaaccatatatctctagccttattgggaaaaaaaataaattcacaacgcaattgttaacggcaagcttacgcttaagtagccagtattcatcttctctttatcaacttatcaggaagcattactctaattttaagaagaaaaattattttattatttccgttgatgagttaaaggaagagttaatagcttatacttttgataaagatggaagtattgagtacaaataccctgactttcctatttttaaaagggatgtattaaataaagccattgctgaaattaaaaagaaaacagaaatatcgtttgttggctttactgttcatgaaaaagaaggaagaaaaattagtaagctgaagttcgaatttgtcgttgatgaagatgaattttctggcgataaagatgatgaagctttttttatgaatttatctgaagctaatgcagcttttctcaaggtatttgatgaaaccgtacctcccaaaaaagctaaggggtgatatatggctaaaatttacgatttccctcaaggagccgaacgccgcaggatgcaccgcaaaatccagtggaacaacgctgtaaaattatctaaaaatggctggagtaagccagaggttaaacgctggtcttttttagcattcatctcaactggctggcggccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtgatacactattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcgcaaactattaactggcgaactacttactctagcttcccggcaacaattaatagactgaatggaggcggataaagttgcaggaccacttctgcgctcggcccttccggctggctggtttattgctgataaatctggagccggtgagcgtgggtctcgcggtatcattgcagcactggggccagatggtaagcgctcccgtatcgtagttatctacaccacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatcgcatgc

SEQ ID NO: 25 - DNA, 1185 bp, Artificial Sequence
  Synthesized
atggcgtccgtaactgtagagcaaatccgaaaggctcagcgagctgaaggtccggccaccatcctcgccattggcaccgccgttcctgccaactgtttcaaccaagctgattttcccgactactactttcgtgtcaccaaaagtgaacacatgactgatctcaaaaagaagttccaacgaatgtgtgaaaaatccactataaaaaagcgttacttgcacttgaccgaagagcatctgaagcagaacccacatctgtgcgagtacaatgcaccatctctgaacacacgccaagacatgttggtggttgaagttcccaagcttgggaaggaggctgcaatcaatgccatcaaagaatggggccaacccaagtccaagatcacccatctcatcttctgcaccggctcctccatcgacatgccaggagccgattaccaatgcgccaagcttctcggcctccgaccctcggtgaagcgagtgatgctgtatcaactcggctgttatgccggtggaaaagttcttcgcatagccaaggacatagcagagaacaacaagggcgctagagttctcattgtgtgctctgagatcacagcttgtatctttcgcgggccctcggagaaacatttggattgcttggtggggcaatctctgttcggagacggggcatcttcggtcatcgttggtgccgaccctgatgcctcggtaggcgagcggccgatcttcgagttggtttcagctgcgcagacgattttgcctaactcggatggagccatagccgggcacgtaacggaagccgggctgacatttcacttgctgagggacgtgccagggttgatctcccaaaacattgagaagagcttgattgaggccttcactccgattgggattaatgactggaacaacatattctggattgcacatcccggtggacctgccattctggacgagatagaggccaagctcgagctgaagaaggagaagatgaaggcgtctcgtgaaatgctgagcgagtatgggaacatgtcatgtgcaagcgttttcttcatagtagatgagatgaggaaacagtcgtcgaaggaagggaagtctaccaccggagatggactggagtggggcgctctcttcgggtttggaccgggtctgacggtggagacggtggtcttgcacagcgtgcccacaaacgtctaa

SEQ ID NO: 26 - protein, 394 aa, Artificial Sequence
  Synthesized
MASVTVEQIRKAQRAEGPATILAIGTAVPANCFNQADFPDYYFRVTKSEHMTDLKKKFQRMCEKSTIKKRYLHLTEEHLKQNPHLCEYNAPSLNTRQDMLVVEVPKLGKEAAINAIKEWGQPKSKITHLIFCTGSSIDMPGADYQCAKLLGLRPSVKRVMLYQLGCYAGGKVLRIAKDIAENNKGARVLIVCSEITACIFRGPSEKHLDCLVGQSLFGDGASSVIVGADPDASVGERPIFELVSAAQTILPNSDGAIAGHVTEAGLTFHLLRDVPGLISQNIEKSLIEAFTPIGINDWNNIFWIAHPGGPAILDEIEAKLELKKEKMKASREMLSEYGNMSCASVFFIVDEMRKQSSKEGKSTTGDGLEWGALFGFGPGLTVETVVLHSVPTNV

SEQ ID NO: 27 - DNA, 954 bp, Artificial Sequence
  Synthesized
atgcgggcgaaacttctgggaatagtcctgacaacccctattgcgatcagctcttttgcttctaccgagactttatcgtttactcctgacaacataaatgcggacattagtcttggaactctgagcggaaaaacaaaagagcgtgtttatctagccgaagaaggaggccgaaaagtcagtcaactcgactggaaattcaataacgctgcaattattaaaggtgcaattaattgggatttgatgccccagatatctatcggggctgctggctggacaactctcggcagccgaggtggcaatatggtcgatcaggactggatggattccagtaaccccggaacctggacggatgaaagtagacaccctgatacacaactcaattatgccaacgaatttgatctgaatatcaaaggctggctcctcaacgaacccaattaccgcctgggactcatggccggatatcaggaaagccgttatagctttacagccagaggtggttcctatatctacagttctgaggagggattcagagatgatatcggctccttcccgaatggagaaagagcaatcggctacaaacaacgttttaaaatgccctacattggcttgactggaagttatcgttatgaagattttgaactcggtggcacatttaaatacagcggctgggtggaatcatctgataacgatgaacactatgacccgggaaaaagaatcacttatcgcagtaaggtcaaagaccaaaattactattctgttgcagtcaatgcaggttattacgtcacacctaacgcaaaagtttatgttgaaggcgcatggaatcgggttacgaataaaaaaggtaatacttcactttatgatcacaataataacacttcagactacagcaaaaatggagcaggtatagaaaactataacttcatcactactgctggtcttaagtacacattttaa

SEQ ID NO: 28 - protein, 317 aa, Artificial Sequence
  Synthesized
MRAKLLGIVLTTPIAISSFASTETLSFTPDNINADISLGTLSGKTKERVYLAEEGGRKVSQLDWKFNNAAIIKGAINWDLMPQISIGAAGWTTLGSRGGNMVDQDWMDSSNPGTWTDESRHPDTQLNYANEFDLNIKGWLLNEPNYRLGLMAGYQESRYSFTARGGSYIYSSEEGFRDDIGSFPNGERAIGYKQRFKMPYIGLTGSYRYEDFELGGTFKYSGWVESSDNDEHYDPGKRITYRSKVKDQNYYSVAVNAGYYVTPNAKVYVEGAWNRVTNKKGNTSLYDHNNNTSDYSKNGAGIENYNFITTAGLKYTF

SEQ ID NO: 29 - DNA, 39 bp, Artificial Sequence
  Synthesized
taacaggaggaattaaccatgacggtcatggagcagcgg

SEQ ID NO: 30 - DNA, 63 bp, Artificial Sequence
  Synthesized
cggtgctggcgtcgttcgccatttaattcctcctgactacagggtgcgcagcagcaccgccgg

SEQ ID NO: 31 - DNA, 43 bp, Artificial Sequence
  Synthesized
gctaacaggaggaattaaccgtgaccgtggagagcactgccgc

SEQ ID NO: 32 - DNA, 64 bp, Artificial Sequence
  Synthesized
cggtgctggcgtcgttcgccatttaattcctcctgactaggcccaggtgatcagccgcttcggc

SEQ ID NO: 33 - DNA, 45 bp, Artificial Sequence
  Synthesized
ttaattcctcctgatcagtacgccagcgagcggtcgacggcatcg

SEQ ID NO: 34 - DNA, 36 bp, Artificial Sequence
  Synthesized
tcaggaggaattaaatggcgaacgacgccagcaccg

SEQ ID NO: 35 - DNA, 57 bp, Artificial Sequence
  Synthesized
tagtaccagatctaccctcgagtcagtcgtgcgagtgcagcggcttgcccgcgaggg

SEQ ID NO: 36 - DNA, 51 bp, Artificial Sequence
  Synthesized
tcgctggcgtactgatcaggaggaattaaatgactgaagcgtccgtgcgtg

SEQ ID NO: 37 - DNA, 43 bp, Artificial Sequence
  Synthesized
ttaattcctcctgattatgcccaagtgatgagacgcttcggct

SEQ ID NO: 38 - DNA, 53 bp, Artificial Sequence
  Synthesized
ctcatcacttgggcataatcaggaggaattaaatggcaaacgacgcatctacg

SEQ ID NO: 39 - DNA, 55 bp, Artificial Sequence
  Synthesized
tagtaccagatctaccctcgagttagtcgtgagaatgcagcggcttgccagccag

SEQ ID NO: 40 - DNA, 20 bp, Artificial Sequence
  Synthesized
cggcgtcacactttgctatg

SEQ ID NO: 41 - DNA, 21 bp, Artificial Sequence
  Synthesized
cgtttcacttctgagttcggc

SEQ ID NO: 42 - DNA, 28 bp, Artificial Sequence
  Synthesized
gattgaagatggtgacctgatcgctatc

SEQ ID NO: 43 - DNA, 27 bp, Artificial Sequence
  Synthesized
gctgtggcgtggcattgttgccggaag

SEQ ID NO: 44 - DNA, 29 bp, Artificial Sequence
  Synthesized
tgtatcggctcgcttcaatccagaaacct

SEQ ID NO: 45 - DNA, 28 bp, Artificial Sequence
  Synthesized
aatttgttcggcgaccagtttaccgaga

SEQ ID NO: 46 - DNA, 28 bp, Artificial Sequence
  Synthesized
atgactgattttttacgcgatgacatca

SEQ ID NO: 47 - DNA, 66 bp, Artificial Sequence
  Synthesized
ccccggggcgattttcacctcggggaaattttagttggcgttcctagccggagttgcgcagcgcag

SEQ ID NO: 48 - DNA, 66 bp, Artificial Sequence
  Synthesized
acatattcaatcattaaaacgattgaatggagaacttttgtctcgagaatatcctccttataactt

SEQ ID NO: 49 - DNA, 66 bp, Artificial Sequence
  Synthesized
gatttgaccgaggaacctgatgtcatcgcgtaaaaaatcagtcatggttaattcctccttccacac

SEQ ID NO: 50 - DNA, 26 bp, Artificial Sequence
  Synthesized
actggaatctgcgaattgtcgccagt

SEQ ID NO: 51 - DNA, 26 bp, Artificial Sequence
  Synthesized
aaaagttctccattcaatcgttttaa

SEQ ID NO: 52 - DNA, 25 bp, Artificial Sequence
  Synthesized
gaacgccaactaaaatttccccgag

SEQ ID NO: 53 - DNA, 26 bp, Artificial Sequence
  Synthesized
aaaagttctccattcaatcgttttaa

SEQ ID NO: 54 - DNA, 25 bp, Artificial Sequence
  Synthesized
ccgtacaccggaagtgttccggcta

SEQ ID NO: 55 - DNA, 45 bp, Artificial Sequence
  Synthesized
gctaacaggaggaattaaccatgggcagcagccatcaccatcatc

SEQ ID NO: 56 - DNA, 45 bp, Artificial Sequence
  Synthesized
actagtaccagatctaccctttacacggtaatcgcccgtccgcga

SEQ ID NO: 57 - DNA, 25 bp, Artificial Sequence
  Synthesized
tagcatttttatccataagattagc

SEQ ID NO: 58 - DNA, 42 bp, Artificial Sequence
  Synthesized
gctaacaggaggaattaaccatggcgtccgtaactgtagagc

SEQ ID NO: 59 - DNA, 48 bp, Artificial Sequence
  Synthesized
tagtaccagatctaccctcgagttagacgtttgtgggcacgctgtgca

SEQ ID NO: 60 - DNA, 46 bp, Artificial Sequence
  Synthesized
ctgacatgcatcatcatcatcatcacgccgagaagatggcgatcgc

SEQ ID NO: 61 - DNA, 22 bp, Artificial Sequence
  Synthesized
catgtcagttacccccctgtcc